SEQUENCE LISTING (13 sequences)

SEQ ID NO: 1 - DNA, 546 bp, synthetic construct
  source 1..546: mol_type = other DNA
gcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcctaa

SEQ ID NO: 2 - DNA, 4247 bp, synthetic construct
  source 1..4247: mol_type = other DNA
ctacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctactaaggtcgccgttgagaaacgcgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcctaagtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttcttaactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagagg

SEQ ID NO: 3 - DNA, 149 bp, synthetic construct
  source 1..149: mol_type = other DNA
gctgataaacagctgacatcaactaaaagtttcattaaatactttgaaaaaagttgttgacttaaaagaagctaaatgttatagtaattgtacagaatagtcttttaagtaagtctactctgaatttttttaaaaggagagggtaaaga

SEQ ID NO: 4 - DNA, 86 bp, synthetic construct
  source 1..86: mol_type = other DNA
gtgagaagcaaaaaattgtggatcagcttgttgtttgcgttaacgttaatctttacgatgggttcagcaacatgtctgctagcgca

SEQ ID NO: 5 - DNA, 546 bp, synthetic construct
  source 1..546: mol_type = other DNA
gcagatccgcctccggttcatgatacagatggccatgaactgagagcagatgcaaactattatgttctgtcagcaaatagagcacatggcggaggcctgacaatggcacctggccatggcagacattgtccgctgtttgtttcacaagatccgaatggccaacatgatggctttccggttagaattacaccgtatggcgttgcaccgtcagataaaatcattagactgagcacagatgtccgcattagctttagagcatatacaacatgcctgcaatcaacggaatggcatattgattcagaactggcagcaggcagacgccatgttattacaggaccggttaaagatccgtcaccgtcaggcagagaaaatgcgtttagaatcgaaaaatacagcggagcggaagtccatgaatataaactgatgtcatgcggagattggtgccaagatctgggcgtttttagagatctgaaaggcggagcatggtttcttggcgcaacagaaccgtatcatgttgtcgttttcaaaaaagcaccgcctgcataa

SEQ ID NO: 6 - DNA, 248 bp, synthetic construct
  source 1..248: mol_type = other DNA
tacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 7 - DNA, 1211 bp, synthetic construct
  source 1..1211: mol_type = other DNA
atgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaacttacctaaaaagcctgagatccctcaggctttttttcctc

SEQ ID NO: 8 - DNA, 43 bp, synthetic construct
  source 1..43: mol_type = other DNA
cctgagatccctcaggctttttttcctcgaaatggatcgaagc

SEQ ID NO: 9 - DNA, 42 bp, synthetic construct
  source 1..42: mol_type = other DNA
catgaaccggaggcggatctgcagcctgcgcagacatgttgc

SEQ ID NO: 10 - DNA, 42 bp, synthetic construct
  source 1..42: mol_type = other DNA
gcaacatgtctgcgcaggctgcagatccgcctccggttcatg

SEQ ID NO: 11 - DNA, 32 bp, synthetic construct
  source 1..32: mol_type = other DNA
cggttttttatgtaagcttatgcaggcggtgc

SEQ ID NO: 12 - DNA, 32 bp, synthetic construct
  source 1..32: mol_type = other DNA
gcaccgcctgcataagcttacataaaaaaccg

SEQ ID NO: 13 - DNA, 43 bp, synthetic construct
  source 1..43: mol_type = other DNA
gcttcgatccatttcgaggaaaaaaagcctgagggatctcagg

(19) United States
(12) Patent Application Publication
(10) Pub. No.: US 2026/0365717 A1
(43) Pub. Date: Nov. 19, 2026

(54) METHODS FOR PRODUCING MONOCLONAL ANTIBODIES

(71) Applicant: Danisco US Inc., Palo Alto, CA (US)

(72) Inventors: Meng Hong Heng, Belmont, CA (US); David A. Estell, San Mateo, CA (US); Steven Sungjin Kim, Fremont, CA (US); Jacob Andrew Latone, San Jose, CA (US); Michael C. Miller, San Francisco, CA (US)

(21) Appl. No.: 18/994,379

(22) PCT Filed: Jul. 14, 2023
(86) PCT No.: PCT/US2023/070192

§ 371 Date: Jan. 14, 2025

Related U.S. Application Data
(60) Provisional application No. 63/389,472, filed on Jul. 15, 2022.

(51) Int. Cl. C07K 16/00; A61K 38/00; C07K 14/81; C12N 1/16; C12N 1/20; C12N 5/00; C12N 5/04; C12N 5/071; C12R 1/125; C12R 1/685; C12R 1/885

(52) U.S. Cl. CPC C07K 16/00; A61K 38/00; C07K 14/8114; C12N 1/16; C12N 1/20; C12N 5/0062; C12N 5/04; C12N 5/0686; C07K 2317/14; C07K 2317/94; C12N 2510/04; C12R 2001/125; C12R 2001/685; C12R 2001/885

(57) ABSTRACT

Provided herein, inter alia, are compositions and methods directed to production of monoclonal antibodies and/or functional fragments thereof which exhibit decreased proteolysis during fermentation, isolation, and purification.

### CROSS REFERENCE TO RELATED APPLICATION

This application claims priority to U.S. Provisional Patent Application No. 63/389,472, filed Jul. 15, 2022, the disclosure of which is herein incorporated by reference in its entirety.

### INCORPORATION BY REFERENCE OF SEQUENCE LISTING

The present application is being filed along with a Sequence Listing in electronic format. The Sequence Listing is provided as a file entitled NB41904-WO-PCT.xml, created 30 Jun. 2023 which is 19,451 bytes in size. The information in the electronic format of the Sequence Listing is incorporated by reference in its entirety.

### FIELD OF THE INVENTION

Provided herein, inter alia, are methods for producing antibodies and antigen-binding fragments thereof having one or more advantages such as, without limitation, decreased proteolytic degradation.

### BACKGROUND

Antibody-based therapeutics have been used successfully to treat a variety of diseases, including cancer and autoimmune/inflammatory disorders. Antibodies also play a role in diagnostics and other medical devices. Large-scale manufacturing of antibodies is hindered due to limits in the amount of host cell protein production (primarily human embryonic kidney cells (HEK) and Chinese Hamster Ovary (CHO) cells), poor thermostability of manufactured antibodies, and susceptibility to proteolytic cleavage during fermentation, processing, and purification. There is thus a need for improved alternative technologies for producing antibody-based biological therapeutics which result in improvements in manufacturability. Ideally, these improved technologies would result in higher yields of antibody thereby facilitating large-scale production at cost and volume targets that will enable their use as traditional therapeutics and diagnostics but also for use as topical and oral products for human and animal health.

The subject matter disclosed herein addresses these needs and provides additional benefits as well.

### SUMMARY

Provided herein, inter alia, is a recombinant cell comprising a) a heterologously expressed barley alpha-amylase subtilisin inhibitor (BASI) polypeptide; and b) a heterologously expressed monoclonal antibody or functional fragment thereof. In some embodiments, the antibody or functional fragment thereof is a therapeutic antibody or functional fragment thereof. In some of any of the embodiments disclosed herein, the cell is a bacterial, fungal, yeast, plant, or mammalian cell. In some embodiments, the cell is a Trichoderma reesei cell or an Aspergillus niger cell. In some embodiments, the cell is a Bacillus subtilis cell. In some embodiments, the cell is a Chinese Hamster Ovary (CHO) or human embryonic kidney (HEK) cell. In some of any of the embodiments disclosed herein, the BASI polypeptide comprises the amino acid sequence of SEQ ID NO:1. In some of any of the embodiments disclosed herein, the antibody or functional fragment thereof exhibits less proteolytic degradation compared to an antibody or functional fragment thereof that is not heterologously co-expressed with a BASI polypeptide. In some embodiments, the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.

In other aspects, provided herein is a fermentation broth comprising any of the recombinant cells disclosed herein.

In further aspects, provided herein is a method for decreasing proteolysis of a heterologously expressed monoclonal antibody or functional fragment thereof comprising culturing a recombinant cell comprising a) a heterologously expressed barley alpha-amylase subtilisin inhibitor (BASI) polypeptide; and b) the heterologously expressed monoclonal antibody or functional fragment thereof under suitable conditions for production of the heterologously expressed antibody or functional fragment thereof and the BASI polypeptide. In some embodiments the method further comprises isolating the antibody or functional fragment thereof. In some of any of the embodiments disclosed herein, the antibody or functional fragment thereof is a therapeutic antibody or functional fragment thereof. In some embodiments, the cell is a bacterial, fungal, yeast, mammalian, or plant cell. In some embodiments, the cell is a Trichoderma reesei cell or an Aspergillus niger cell. In some embodiments, the cell is a Bacillus subtilis cell. In some embodiments, the cell is a Chinese Hamster Ovary (CHO) or human embryonic kidney (HEK) cell. In some of any of the embodiments disclosed herein, the BASI polypeptide comprises the amino acid sequence of SEQ ID NO:1. In some embodiments, the antibody or functional fragment thereof exhibits less proteolytic degradation compared to an antibody or functional fragment thereof that is not heterologously co-expressed with a BASI polypeptide. In some of any of the embodiments disclosed herein, the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.

In another aspect, provided herein is a method for decreasing proteolysis of a recombinantly expressed monoclonal antibody or functional fragment thereof comprising isolating the recombinantly expressed antibody or functional fragment thereof in the presence of an exogenously added barley alpha-amylase subtilisin inhibitor (BASI) polypeptide. In some embodiments, the antibody or functional fragment thereof is a therapeutic antibody or functional fragment thereof. In some of any of the embodiments disclosed herein, the BASI polypeptide is recombinantly expressed in a bacterial, fungal, yeast, mammalian, or plant cell. In some embodiments, the BASI polypeptide is recombinantly expressed in a Bacillus subtilis cell or an Aspergillus niger cell. In some of any of the embodiments disclosed herein, the BASI polypeptide comprises the amino acid sequence of SEQ ID NO:1. In some embodiments, the monoclonal antibody or functional fragment thereof is recombinantly expressed in a bacterial, fungal, yeast, mammalian, or plant cell. In some embodiments, the monoclonal antibody or functional fragment thereof is recombinantly expressed in a Trichoderma reesei cell or an Aspergillus niger cell. In some embodiments, the monoclonal antibody or functional fragment thereof is recombinantly expressed in a Bacillus subtilis cell. In some embodiments, the monoclonal antibody or functional fragment thereof is recombinantly expressed in a Chinese Hamster Ovary (CHO) or human embryonic kidney (HEK) cell. In some of any of the embodiments disclosed herein, the antibody or functional fragment thereof exhibits less proteolytic degradation and/or improved yields of intact protein compared to an antibody or functional fragment thereof that is not isolated in the presence of an exogenously added BASI polypeptide. In some embodiments, the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts DNAs used to introduce assembled cassettes at the (A & B) I1 and (C & D) 1A loci. For each strain, A or B was combined with C or D for transformation. Pairs of DNAs depicted in A-D each introduce HC and LC for one antibody at one locus. A and D introduce only the antibody expression cassettes. B also introduces the BASI expression cassette. C also introduces the accessory protein cassette.

FIG. 2 is a graph depicting the positive effect of BASI co-expression on the titer of a 150 kDa antibody species (size exclusion chromatography (SEC), right axis) expressed in a strain of T. reesei (BASI 1 SP1). The protein A based titer and SEC based titer for the BASI negative strain (sp1) appear to reach a maximum early and then drop as the fermentation run progresses.

FIG. 3 depicts a 4-12% SDS-PAGE reducing gel. First panel on left is molecular weight marker in kd, second panel from left shows samples stored at −20 °C, third panel from left shows samples stored at 5 °C for 7 days and panel on right shows samples stored at 5 °C for 28 days. Lanes labelled with 1=No BASI; 2=0.5 g BASI Filtrate/g-broth; 3=1 gBASI Filtrate/g-broth; and 4=3 gBASI Filtrate/g-broth.

FIG. 4 depicts a 4-12% SDS-PAGE reducing gel. First panel on left is molecular weight marker in kDa, second panel from left shows samples stored at −20 °C, third panel from left shows samples stored at 22 °C for 7 days and panel on right shows samples stored at 22 °C for 28 days. Lanes labelled with 1=No BASI, 2=0.5 gBASI Filtrate/g-broth, 3=1 gBASI Filtrate/g-broth and 4=3 gBASI Filtrate/g-broth.

FIG. 5 depicts a 4-12% SDS-PAGE reducing gel. The top box depicts samples from complex media and the bottom box depicts samples from defined media. Gel images are arranged in the same order for each picture. Description from left to right: Left lane=molecular weight maker; 2nd lane from left shows sample stored at −20 °C; 3rd and 4th lanes from left are samples stored at 5 °C, with and without BASI addition, respectively; 5th and 6th lanes from left are samples stored at 22 °C, with and without BASI addition, respectively.

FIG. 6 depicts a 4-12% SDS-PAGE reducing gel. Gel image on left=molecular weight marker. Gel images are grouped in box by storage temperature. Left box from −20 °C, middle box from 5 °C, and right box from 22 °C. None=sample with no BASI addition; 1=sample with κ gBASI filtrate per 10 g UFC; 2=sample with 20 gBASI filtrate per 10 g UFC.

FIG. 7 depicts a 4-12% SDS-PAGE reducing gel. Gel image on left=molecular weight marker. Gel images of samples are grouped by storage temperature and time. On the left are from −20 °C. The 2nd group from left are from 22 °C storage after 2 days. The 3rd group from left are from 5 °C storage after 4 days. The group on the right are from 5 °C storage for 10 days. Sample description: None=no BASI addition; M=1 gBASI/kg UFC; L=0.25 gBASI/kg UFC; and H=1.9 gBASI/kg UFC.

FIG. 8 depicts a 4-12% SDS-PAGE reducing gel. Gel on left shows samples from pH 5.5 fermentation with no BASI added and with BASI added taken at fermentation times at 40 h, 70 h, 91 h, 116 h, and 140 h. Gel on the right shows comparable samples from pH 6.5 fermentations.

FIG. 9 depicts a 4-12% SDS-PAGE reducing gel with samples after 15 days storage at 10 °C compared to −20 °C reference. Gel image on left=molecular weight marker; gel image in center=samples with no BASI addition; gel image on right=samples with BASI addition.

FIGS. 10A and 10B depict reducing gels FIG. 10A depicts a 4-12% SDS-PAGE reducing gel comparing samples taken during recovery to a sample of the fermentation broth. Lane 1=purified antibody; 2=end of fermentation broth supernatant; 3=cooled broth supernatant; 4=transferred broth supernatant; 5=treated broth supernatant; 6=cell separated filtrate; 7=filtrate at start of ultrafiltration; 8=ultrafiltration concentrate. FIG. 10B depicts a 4-12% SDS-PAGE reducing gel comparing UF concentrate stored at 10 °C for 1 month to −20 °C.

FIG. 11 depicts a non-reducing SDS-PAGE gel comparing samples of Antibody B from UFC 20200577 formulated to 1 g antibody/L. Samples are from before (lanes 2,4,6,8; stored at −20 °C) and after (lanes 3,5,7,9) storage for 56 days at 4 °C. Lane 1 contains Novex Sharp Pre-Stained Protein Standard and Lane 10 contains CHO-expressed purified antibody standard, 0.5 ug. Lanes 2 and 3 show samples stored in Formulation A (100 mM Bis_Tris, pH 5.8) with residual band density of 7.1%. Lanes 4 and 5 show samples stored in Formulation B (100 mM Bis_Tris, pH 5.8+3 g/kg BASI) with residual band density of 45.2%. Lanes 6 and 7 show samples stored in Formulation C (100 mM Bis_Tris, pH 5.8+2.3% Arginine-HCl) with residual band density of 27.9%. Lanes 8 and 9 show samples stored in Formulation D (100 mM Bis_Tris, pH 5.8+2.3% Arginine-HCl+3 g/L BASI) with residual band density of 79.8%.

FIG. 12 depicts a non-reducing SDS-PAGE gel comparing samples of Antibody B from UFC 20208053 formulated to 1 g antibody/L. Samples are from before (lanes 2,4,6,8; stored at −20 °C) and after (lanes 3,5,7,9) storage for 25 days at 22 °C. Lane 1 contains Novex Sharp Pre-Stained Protein Standard and Lane 10 contains CHO-expressed purified antibody standard, 0.5 ug. Lanes 2 and 3 show samples stored in Formulation A (100 mM Bis_Tris, pH 5.8) with residual band density of 0.7%. Lanes 4 and 5 show samples stored in Formulation B (100 mM Bis_Tris, pH 5.8+3 g/kg BASI) with residual band density of 7.6%. Lanes 6 and 7 show samples stored in Formulation C (100 mM Bis_Tris, pH 5.8+2.3% Arginine-HCl) with residual band density of 4.6%. Lanes 8 and 9 show samples stored in Formulation D (100 mM Bis_Tris, pH 5.8+2.3% Arginine-HCl+3 g/L BASI) with residual band density of 22.6%.

FIG. 13 depicts a non-reducing SDS-PAGE gel comparing samples of Antibody A from UFC 20200874 formulated to 1 g antibody/L. Samples are from before (lanes 2,4,6,8; stored at −20 °C) and after (lanes 3,5,7,9) storage for 25 days at 22 °C. Lane 1 contains Novex Sharp Pre-Stained Protein Standard and Lane 10 contains CHO-expressed purified antibody standard, 0.5 ug. Lanes 2 and 3 show samples stored in Formulation C (100 mM Bis_Tris, pH 5.8+2.3% Arginine-HCl) with residual band density of 0%. Lanes 4 and 5 show samples stored in Formulation D (100 mM Bis_Tris, pH 5.8+2.3% Arginine-HCl+3 g/L BASI) with residual band density of 14.6%. Lanes 6 and 7 show samples stored in Formulation A (100 mM Bis_Tris, pH 5.8) with residual band density of 0%. Lanes 8 and 9 show samples stored in Formulation B (100 mM Bis_Tris, pH 5.8+3 g/kg BASI) with residual band density of 1.6%.

### DETAILED DESCRIPTION

Conventional monoclonal antibody production in mammalian-derived cells, such as Chinese hamster ovary (CHO) or human embryonic kidney (HEK) cells is limited by the titer of product able to be produced by these cells in a single fermentation. Production of monoclonal antibodies in non-mammalian cells, such as filamentous fungal-based cell expression platforms, hold the potential to substantially improve the overall titer and manufacturability of monoclonal antibodies for large and/or industrial scale production. However, these non-mammalian expression platforms are limited by the existence of substantial proteolytic degradation of recombinantly expressed monoclonal antibodies, or functional fragments thereof, during the fermentation, isolation, and purification of the antibodies.

As will be described in more detail herein, the inventors of the present application have surprisingly discovered that a protein inhibitor isolated from barley, the barley alpha-amylase subtilisin inhibitor (BASI), substantially decreases the amount of proteolytic degradation of recombinantly expressed monoclonal antibodies in non-mammalian cell expression systems when co-expressed with the antibody or when exogenously added to the fermentation medium during production. Thus, the use of BASI in the compositions and methods disclosed herein brings about substantially improved titers of intact monoclonal antibodies produced in these non-mammalian systems by markedly decreasing proteolysis of the recombinantly expressed monoclonal antibodies.

### I. DEFINITIONS

As used herein, “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies include antibody fragments. As used herein, the term antibody, thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, such as, but not limited to, Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

As used herein, an “antibody fragment” or “antigen-binding fragment” of an antibody refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically, e.g. recombinantly produced derivatives. An antibody fragment is included among antibodies. Examples of antibody fragments include, but are not limited to, Fab, Fab′, F(ab′)₂, single-chain Fv (scFv), Fv, dsFv, diabody, Fd and Fd′ fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least or about 50 amino acids and typically at least or about 200 amino acids. An antigen-binding fragment includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen.

As used herein, a “therapeutic antibody” refers to any antibody or antigen-binding fragment thereof that is administered for treatment of an animal, including a human. Such antibodies can be prepared by any known methods for the production of polypeptides, and hence, include, but are not limited to, recombinantly produced antibodies, synthetically produced antibodies, and therapeutic antibodies extracted from cells or tissues and other sources. As isolated from any sources or as produced, therapeutic antibodies can be heterogeneous in length or differ in post-translational modification, such as glycosylation (i.e. carbohydrate content). Heterogeneity of therapeutic antibodies also can differ depending on the source of the therapeutic antibodies. Hence, reference to therapeutic antibodies refers to the heterogeneous population as produced or isolated. When a homogeneous preparation is intended, it will be so-stated. References to therapeutic antibodies herein are to their monomeric, dimeric or other multimeric forms, as appropriate.

As used herein, a “neutralizing antibody” is any antibody or antigen-binding fragment thereof that binds to a pathogen and interferes with the ability of the pathogen to infect a cell and/or cause disease in a subject. Exemplary of neutralizing antibodies are neutralizing antibodies that bind to viruses, bacteria, and fungal pathogens. Typically, the neutralizing antibodies provide herein bind to the surface of the pathogen. In examples where the pathogen is a virus, a neutralizing antibody that binds to the virus typically binds to a protein on the surface of the virus. Depending on the class of the virus, the surface protein can be a capsid protein (e.g. a capsid protein of a non-enveloped virus) or a viral envelope protein (e.g., a viral envelope protein of an enveloped virus). In some examples, the protein is a glycoprotein. The ability of the virus to inhibit virus infectivity can be measure for example, by an in vitro neutralization assay, such as, for example, a plaque reduction assay using Vero host cells.

As used herein, a “conventional antibody” refers to an antibody that contains two heavy chains (which can be denoted H and H′) and two light chains (which can be denoted L and L′) and two antibody combining sites, where each heavy chain can be a full-length immunoglobulin heavy chain or any functional region thereof that retains antigen-binding capability (e.g. heavy chains include, but are not limited to, VH, chains VH-CH1 chains and VH-CH1-CH2-CH3 chains), and each light chain can be a full-length light chain or any functional region of (e.g. light chains include, but are not limited to, VL chains and VL-CL chains). Each heavy chain (H and H′) pairs with one light chain (L and L′, respectively)

As used herein, a “full-length antibody” is an antibody having two full-length heavy chains (e.g. VH-CH1-CH2-CH3 or VH-CH1-CH2-CH3-CH4) and two full-length light chains (VL-CL) and hinge regions, such as human antibodies produced naturally by antibody secreting B cells and antibodies with the same domains that are synthetically produced.

As used herein, the phrase “derived from” when referring to antibody fragments derived from another antibody, such as a monoclonal antibody, refers to the engineering of antibody fragments (e.g., Fab, F(ab′), F(ab′)₂, single-chain Fv (scFv), Fv, dsFv, diabody, Fd and Fd′ fragments) that retain the binding specificity of the original antibody. Such fragments can be derived by a variety of methods known in the art, including, but not limited to, enzymatic cleavage, chemical crosslinking, recombinant means or combinations thereof. Generally, the derived antibody fragment shares the identical or substantially identical heavy chain variable region (VH) and light chain variable region (VL) of the parent antibody, such that the antibody fragment and the parent antibody bind the same epitope

As used herein, a “parent antibody” or “source antibody” refers the to an antibody from which an antibody fragment (e.g., Fab, F(ab′), F(ab′)₂, single-chain Fv (scFv), Fv, dsFv, diabody, Fd and Fd′ fragments) is derived.

As used herein, the term “epitope” refers to any antigenic determinant on an antigen to which the paratope of an antibody binds. Epitopic determinants typically contain chemically active surface groupings of molecules such as amino acids or sugar side chains and typically have specific three-dimensional structural characteristics, as well as specific charge characteristics.

As used herein, a “chimeric polypeptide” refers to a polypeptide that contains portions from at least two different polypeptides or from two non-contiguous portions of a single polypeptide. Thus, a chimeric polypeptide generally includes a sequence of amino acid residues from all or part of one polypeptide and a sequence of amino acids from all or part of another different polypeptide. The two portions can be linked directly or indirectly and can be linked via peptide bonds, other covalent bonds or other non-covalent interactions of sufficient strength to maintain the integrity of a substantial portion of the chimeric polypeptide under equilibrium conditions and physiologic conditions, such as in isotonic pH 7 buffered saline. For purposes herein, chimeric polypeptides include those containing all or part of an antibody or functional fragment thereof and/or BASI polypeptide linked to another polypeptide, such as, for example, a multimerization domain, a heterologous immunoglobulin constant domain or framework region, a diagnostic or therapeutic polypeptide, or a secretion enhancing polypeptide.

As used herein, a “fusion protein” is a polypeptide engineered to contain sequences of amino acids corresponding to two distinct polypeptides, which are joined together, such as by expressing the fusion protein from a vector containing two nucleic acids, encoding the two polypeptides, in close proximity, e.g., adjacent, to one another along the length of the vector. Generally, a fusion protein provided herein refers to a polypeptide that contains a polypeptide having the amino acid sequence of an antibody or antigen-binding fragment thereof and a polypeptide or peptide having the amino acid sequence of a heterologous polypeptide or peptide, such as, for example, a diagnostic or therapeutic polypeptide. Accordingly, a fusion protein refers to a chimeric protein containing two, or portions from two, or more proteins or peptides that are linked directly or indirectly via peptide bonds. The two molecules can be adjacent in the construct or separated by a linker, or spacer polypeptide. The spacer can encode a polypeptide that alters the properties of the polypeptide, such as solubility or intracellular trafficking.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary of polypeptide linkers are (Gly-Ser) amino acid sequences, with some Glu or Lys residues dispersed throughout to increase solubility. Other exemplary linkers are described herein; any of these and other known linkers can be used with the provided compositions and methods.

As used herein, “antibody hinge region” or “hinge region” refers to a polypeptide region that exists naturally in the heavy chain of the gamma, delta, and alpha antibody isotypes, between the CH1 and CH2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility allows the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge regions, for example, to promote stability via interactions between two antibody chains. Hinge regions are exemplary of dimerization domains.

As used herein, “humanized antibodies” refer to antibodies that are modified to include “human” sequences of amino acids so that administration to a human does not provoke an immune response. A humanized antibody typically contains complementarily determining regions (CDRs) derived from a non-human species immunoglobulin and the remainder of the antibody molecule derived mainly from a human immunoglobulin. Methods for preparation of such antibodies are known. For example, DNA encoding a monoclonal antibody can be altered by recombinant DNA techniques to encode an antibody in which the amino acid composition of the non-variable regions is based on human antibodies. Methods for identifying such regions are known, including computer programs, which are designed for identifying the variable and non-variable regions of immunoglobulins.

As used herein, an “Ig domain” is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (VL) and one constant region domain (CL), while a heavy chain contains one variable region domain (VH) and three or four constant region domains (CH). Each VL, CL, VH, and CH domain is an example of an immunoglobulin domain.

As used herein, a “variable domain” or “variable region” is a specific Ig domain of an antibody heavy or light chain that contains a sequence of amino acids that varies among different antibodies. Each light chain and each heavy chain has one variable region domain, VL and VH, respectively. The variable domains provide antigen specificity, and thus are responsible for antigen recognition. Each variable region contains CDRs that are part of the antigen-binding site domain and framework regions (FRs).

As used herein, “antigen-binding domain,” “antigen-binding site,” “antigen combining site” and “antibody combining site” are used synonymously to refer to a domain within an antibody that recognizes and physically interacts with cognate antigen. A native conventional full-length antibody molecule has two conventional antigen-binding sites, each containing portions of a heavy chain variable region and portions of a light chain variable region. A conventional antigen-binding site contains the loops that connect the anti-parallel beta strands within the variable region domains. The antigen combining sites can contain other portions of the variable region domains. Each conventional antigen-binding site contains three hypervariable regions from the heavy chain and three hypervariable regions from the light chain. The hypervariable regions also are called complementarity-determining regions (CDRs).

As used herein, “hypervariable region,” “HV,” “complementarity-determining region” and “CDR” and “antibody CDR” are used interchangeably to refer to one of a plurality of portions within each variable region that together form an antigen-binding site of an antibody. Each variable region domain contains three CDRs, named CDR1, CDR2 and CDR3. The three CDRs are non-contiguous along the linear amino acid sequence but are proximate in the folded polypeptide. The CDRs are located within the loops that join the parallel strands of the beta sheets of the variable domain. As described herein, one of skill in the art knows and can identify the CDRs and framework regions based on Kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, the disclosures of which are incorporated by reference herein).

As used herein, “framework regions” (FRs) are the domains within the antibody variable region domains that are located within the beta sheets; the FR regions are comparatively more conserved, in terms of their amino acid sequences, than the hypervariable regions.

As used herein, a “constant region” or “constant domain” is a domain in an antibody heavy or light chain that contains a sequence of amino acids that is comparatively more conserved than that of the variable region domain. In conventional full-length antibody molecules, each light chain has a single light chain constant region (CL) domain and each heavy chain contains one or more heavy chain constant region (CH) domains, which include, CH1, CH2, CH3 and CH4. Full-length IgA, IgD and IgG isotypes contain CH1, CH2, CH3 and a hinge region, while IgE and IgM contain CH1, CH2, CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g., through interactions with various cells, biomolecules and tissues.

As used herein, a functional region of an antibody is a portion of the antibody that contains at least a VH, VL, CH (e.g. CH1, CH2 or CH3), CL or hinge region domain of the antibody, or at least a functional region thereof.

As used herein, “specifically bind” or “immunospecifically bind” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen. Affinity constants can be determined by standard kinetic methodology for antibody reactions, for example, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989)). Instrumentation and methods for real time detection and monitoring of binding rates are known and are commercially available (e.g., BiaCore 2000, Biacore AB, Upsala, Sweden and GE Healthcare Life Sciences; Malmqvist (2000) Biochem. Soc. Trans. 27:335). Typically, an antibody or antigen-binding fragment thereof provided herein that binds immunospecifically to an epitope does not cross-react with other antigens or cross reacts with substantially (at least 10-100 fold) lower affinity for such antigens. Antibodies or antigen-binding fragments that immunospecifically bind to a particular epitope can be identified, for example, by immunoassays, such as radioimmunoassays (RIA), enzyme-linked immunosorbent assays (ELISAs), surface plasmon resonance, or other techniques known to those of skill in the art. An antibody or antigen-binding fragment thereof that immunospecifically binds to an epitope typically is one that binds to the epitope with a higher binding affinity than to any cross-reactive epitope as determined using experimental techniques, such as, but not limited to, immunoassays, surface plasmon resonance, or other techniques known to those of skill in the art. Immunospecific binding to an isolated protein (i.e., a recombinantly produced protein), does not necessarily mean that the antibody will exhibit the same immunospecific binding. Such measurements and properties are distinct. The affinity for the antibody or antigen-binding fragments for the antigen as presented can be determined. For purposes herein, when describing an affinity or related term, the target, such as the isolated protein, will be identified.

As used herein, “Fc” or “Fc region” or “Fc domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG, Fc contains immunoglobulin domains Cγ2 and Cγ3, and optionally, all or part of the hinge between Cγ1 and Cγ2. The boundaries of the Fc region can vary, but typically, include at least part of the hinge region. In addition, Fc also includes any allelic or species variant or any variant or modified form, such as any variant or modified form that alters the binding to an FcR or alters an Fc-mediated effector function.

As used herein, a “tag” or an “epitope tag” refers to a sequence of amino acids, typically added to the N- or C-terminus of a polypeptide, such as an antibody provided herein. The inclusion of tags fused to a polypeptide can facilitate polypeptide purification and/or detection. Typically, a tag or tag polypeptide refers to polypeptide that has enough residues to provide an epitope recognized by an antibody or can serve for detection or purification yet is short enough such that it does not interfere with activity of chimeric polypeptide to which it is linked. The tag polypeptide typically is sufficiently unique so an antibody that specifically binds thereto does not substantially cross-react with epitopes in the polypeptide to which it is linked. Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well known and can be readily synthesized and designed. Exemplary tag polypeptides include those used for affinity purification and include, His tags, the influenza hemagglutinin (HA) tag polypeptide and its antibody 12CA5, (Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5:3610-3616); and the Herpes Simplex virus glycoprotein D (gD) tag and its antibody (Paborsky et al. (1990) Protein Engineering 3:547-553). An antibody used to detect an epitope-tagged antibody is typically referred to herein as a secondary antibody.

As used herein, “polypeptide” refers to two or more amino acids covalently joined. The terms “polypeptide” and “protein” are used interchangeably herein.

As used herein, a “peptide” refers to a polypeptide that is from 2 to about or 40 amino acids in length.

The “barley α-amylase/subtilisin inhibitor” or “BASI” is a double headed inhibitor of α-amylase from barley and on serine proteases of the subtilisin family. In some embodiments, BASI comprises the amino acid sequence of SEQ ID NO:1. In other embodiments, BASI may have at least about 50% identity to the mature peptide of SEQ ID NO: 1 or it may comprise a sequence having at least about 50% identity to residues 67-96 of SEQ ID NO: 1. The identity may particularly be at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99%, identical to SEQ ID NO:1 or to residues 67-96 of SEQ ID NO: 1.

As used herein, an “amino acid” is an organic compound containing an amino group and a carboxylic acid group. A polypeptide contains two or more amino acids. For purposes herein, amino acids contained in the antibodies provided include the twenty naturally-occurring amino acids, non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations. The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§ 1.821-1.822, abbreviations for amino acid residues are used throughout. All sequences of amino acid residues represented herein by a formula have a left to right orientation in the conventional direction of amino-terminus to carboxyl-terminus. In addition, the phrase “amino acid residue” is defined to include natural, modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH. As used herein, “naturally occurring amino acids” refer to the 20 L-amino acids that occur in polypeptides.

As used herein, “Stability” and “stable” refer to the resistance of antibodies in a formulation to aggregation, degradation (such as proteolytic degradation) or fragmentation (such as proteolytic-mediated fragmentation) under given manufacture, preparation, transportation and storage conditions. An antibody with improved stability and resistance to degradation will retain biological activity under given manufacture, preparation, transportation and storage conditions. The stability of an antibody can be assessed by degrees of aggregation, degradation or fragmentation, as measured by High Performance Size Exclusion Chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques. Stability of an antibody can also be measured by assessment of the intensity of a band on a gel representative of the full-length antibody species both before and after treatment. The stability of an antibody may be compared to a comparable molecule under identical conditions. The overall stability of an antibody can also be assessed by various immunological assays including, for example, ELISA and radioimmunoassay using isolated antigen molecules or cells expressing the same.

As used herein, an “activity” or a “functional activity” of a polypeptide, such as an antibody, refers to any activity exhibited by the polypeptide. Such activities can be empirically determined. Exemplary activities include, but are not limited to, ability to interact with a biomolecule, for example, through antigen-binding, DNA binding, ligand binding, or dimerization, enzymatic activity, for example, kinase activity or proteolytic activity. For an antibody (including antibody fragments), activities include, but are not limited to, the ability to specifically bind a particular antigen, affinity of antigen-binding (e.g. high or low affinity), avidity of antigen-binding (e.g. high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body or improved manufacturability, thermostability, or protease resistance. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g., panning assays). For example, for an antibody polypeptide, activities can be assessed by measuring binding affinities, avidities, and/or binding coefficients (e.g., for on-/off-rates), and other activities in vitro or by measuring various effects in vivo, such as immune effects, e.g. antigen clearance, penetration or localization of the antibody into tissues, protection from disease, e.g. infection, serum or other fluid antibody titers, or other assays that are well known in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (e.g. variant) antibodies are well known in the art.

As used herein, “exhibits less proteolytic degradation” refers to the degree of proteolytic cleavage of a recombinantly produced antibody or functional fragment thereof during fermentation, isolation, purification, and/or storage, such as an antibody produced according to the methods provided herein, such as a therapeutic antibody or functional fragment thereof, compared to an identical antibody that is not produced in accordance with the methods provided herein. Antibodies or functional fragments thereof produced in accordance with the methods provided herein can exhibit any of about 1% less proteolytic degradation, 2%, 3%, 4%, 5%, 10%, 20%, 30%, 40%, 50%, 60%, 70%, 80%, 90%, 91%, 92%, 93%, 94%, 95%, 96%, 97%, 98%, 99%, or 100% less proteolytic degradation compared to antibodies or functional fragments thereof that are not produced in accordance with the methods disclosed herein. In other embodiments, the decrease in proteolytic degradation is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more less. Assays for determining the extent of proteolytic degradation of a subject antibody or functional fragment thereof are well known in the art. Such assays can be performed in vitro or in vivo. Activity can be measured, for example, using assays described in the Examples below.

As used herein, “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

As used herein, “regulatory region” of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, “production by recombinant means by using recombinant DNA methods” means the use of the well-known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids. In one example, the host cell is a genetic package, which can be induced to express the variant polypeptide on its surface. In another example, the host cell is infected with the genetic package. For example, the host cells can be phage-display compatible host cells, which can be transformed with phage or phagemid vectors and accommodate the packaging of phage expressing fusion proteins containing the variant polypeptides.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “similarity” between two proteins or nucleic acids refers to the relatedness between the sequence of amino acids of the proteins or the nucleotide sequences of the nucleic acids. Similarity can be based on the degree of identity of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, when a polypeptide or nucleic acid molecule or region thereof contains or has “identity” or “homology” to another polypeptide or nucleic acid molecule or region, the two molecules and/or regions share greater than or equal to at or about 40% sequence identity, and typically greater than or equal to at or about 50% sequence identity, such as at least or about 60%, 65%, 70%, 75%, 80%, 85%, 90%, 95%, 96%, 97%, 98%, 99% or 100% sequence identity; the precise percentage of identity can be specified if necessary. A nucleic acid molecule, or region thereof, that is identical or homologous to a second nucleic acid molecule or region can specifically hybridize to a nucleic acid molecule or region that is 100% complementary to the second nucleic acid molecule or region. Identity alternatively can be compared between two theoretical nucleotide or amino acid sequences or between a nucleic acid or polypeptide molecule and a theoretical sequence.

Sequence “identity,” per se, has an art-recognized meaning and the percentage of sequence identity between two nucleic acid or polypeptide molecules or regions can be calculated using published techniques. Sequence identity can be measured along the full length of a polynucleotide or polypeptide or along a region of the molecule. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., SIAM J Applied Math 48:1073 (1988)).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions is 100%. Alternatively, sequence identity can be compared along the length of a molecule, compared to a region of another molecule. Alternatively, sequence identity between polypeptide A and polypeptide B can be compared along the same length polypeptide but with amino acid replacements, such as conservative amino acid replacements or non-conservative amino acid replacements. As discussed below, and known to those of skill in the art, various programs and methods for assessing identity are known to those of skill in the art. High levels of identity, such as 90% or 95% identity, readily can be determined without software.

Whether any two nucleic acid or polypeptide molecules have nucleotide sequences that are at least or about 60%, 70%, 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85:2444 (other programs include the GCG program package (Devereux, J. et al. (1984) Nucleic Acids Research 12 (I): 387), BLASTP, BLASTN, FASTA (Altschul, S. F. et al. (1990) J. Molec. Biol. 215:403; Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, WI) and the University of Wisconsin Genetics Computer Group (UWG) “Gap” program (Madison WI)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

As used herein, a “modification” is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, “substitution” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence.

As used herein, a “label” or “detectable moiety” is a detectable marker (e.g., a fluorescent molecule, chemiluminescent molecule, a bioluminescent molecule, a contrast agent (e.g., a metal), a radionuclide, a chromophore, a detectable peptide, or an enzyme that catalyzes the formation of a detectable product) that can be attached or linked directly or indirectly to a molecule or associated therewith and can be detected in vivo and/or in vitro. The detection method can be any method known in the art, including known in vivo and/or in vitro methods of detection (e.g., imaging by visual inspection, magnetic resonance (MR) spectroscopy, ultrasound signal, X-ray, gamma ray spectroscopy (e.g., positron emission tomography (PET) scanning, single-photon emission computed tomography (SPECT)), fluorescence spectroscopy or absorption). Indirect detection refers to measurement of a physical phenomenon, such as energy or particle emission or absorption, of an atom, molecule or composition that binds directly or indirectly to the detectable moiety (e.g., detection of a labeled secondary antibody or antigen-binding fragment thereof that binds to a primary antibody.

As used herein, an “isolated” or “purified” polypeptide or protein (e.g. an isolated antibody or antigen-binding fragment thereof) or biologically-active portion thereof (e.g. an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound. As used herein, a “cellular extract” or “lysate” refers to a preparation or fraction which is made from a lysed or disrupted cell.

As used herein, “oxygen uptake rate” or “OUR” refers to the rate of oxygen consumption in the bioreactor defined as mmol O₂/L of broth/h (Buckland et al., 1985).

As used herein, the phrase “dissolved oxygen” or “DO %” refers to the amount of oxygen dissolved in the broth and is expressed in percent of air saturation.

As used herein, “carbon dioxide evolution rate” or “CER” refers to the rate of carbon dioxide produced in the bioreactor defined as mmol CO₂/L of broth/h (Buckland et al., 1985).

As used herein, “elapsed fermentation time” or “EFT” refers to the number of hours that have passed since the bioreactor was inoculated with the seed culture.

“Seed culture,” as used herein, refers to a liquid culture used to build up the biomass required for starting the production culture.

“Dry cell weight” or “DCW,” as used herein, refers to the grams of dry weight per kilogram of broth, and will be used as a measure of biomass accumulation.

The term “biomass,” as used herein, refers to the total mass of a recombinant host cell strain (e.g. a fungal strain) in a defined volume.

As used herein, the phrase “specific sugar feed rate” or “Qs” refers to the rate at which sugar is fed divided by the amount of biomass in the fermenter.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular terms “a,” “an,” and “the” include the plural reference unless the context clearly indicates otherwise.

As used herein, “optional” or “optionally” means that the subsequently circumstance or limitation on scope does or does not occur, and that the description includes instances where the circumstance or limitation on scope occurs and instances where it does not. For example, in a composition that optionally contains additional exogenous enzymes means that the enzymes can be present or not present in the composition.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. COMPOSITIONS

### A. BASI

The barley alpha-amylase subtilisin inhibitor (BASI) polypeptide belongs to the Kunitz-type trypsin inhibitor family (Leah & Mundy, 1989, Plant Mol. Biol. 12, 673-682). BASI is a single chain protein consisting of 181 amino acids and contains two disulphide bridges, which are conserved in the structure of Kunitz inhibitors. BASI shares 92 and 58% sequence identity with analogous inhibitors from wheat (WASI) and rice (RASI) (Mundy et al., 1984, FEBS Lett. 167, 210-214; Ohtsubo & Richardson, 1992, FEBS Lett. 309, 68-72), respectively (Micheelsen et al., 2008, Journal of Biotechnology 133 (2008) 424-432).

The inventors of the present application have discovered that when used in a monoclonal antibody (or functional fragment thereof) production process, BASI reduces protease activity, resulting in higher yield of intact protein, lower yield of degraded protein, and improved performance in downstream protease-sensitive applications. BASI can be used by recombinant co-expression with a monoclonal antibody or functional fragment thereof or via addition of exogenous BASI (or BASI-containing fermentation broth) to fermentation broth, recovered protein solution, or a formulated monoclonal antibody (or functional fragment thereof) product.

In some embodiments, BASI comprises the amino acid sequence of SEQ ID NO:1. In other embodiments, BASI may have at least about 50% identity to the mature peptide of SEQ ID NO: 1 or it may comprise a sequence having at least about 50% identity to residues 67-96 of SEQ ID NO: 1. The identity may particularly be at least about 60%, at least about 70%, at least about 80%, at least about 85%, at least about 90%, at least about 95% or at least about 98% identical to SEQ ID NO: 1 or to residues 67-96 of SEQ ID NO: 1.

### B. Antibodies

Provided herein are antibodies, fragments thereof, or variants thereof whose co-expression or co-fermentation with BASI results in decreased cleavage (such as proteolytic cleavage) and/or increased titers or fermentation yields. The recombinant monoclonal antibodies disclosed herein may be generated by methods well known in the art.

An antibody or functional fragment thereof produced by the methods disclosed herein may also display one or more of improved purification yields; reduced formulation problems; reduced immunogenicity and increased bioavailability relative to the same antibody or functional fragment thereof that is not produced in accordance with the methods disclosed herein. In some embodiments, improvements in manufacturability may result from both reduced proteolysis-mediated aggregation-propensity and increased productivity due to decreased protein cleavage and degradation.

The antibodies or functional fragments thereof produced in accordance with the methods disclosed herein exhibit increased or improved or enhanced protease resistance compared to an antibody or functional fragment thereof that is not produced in accordance with the methods disclosed herein. The term “protease resistance” refers to the ability of a molecule comprised of peptide bonds, to resist hydrolytic cleavage of one or more of its peptide bonds in the presence of a proteolytic enzyme. The resistance to proteolytic enzymes is a relative property and is compared to a molecule (such as a molecule not produced in accordance with the methods disclosed herein, such as by co-expression or co-fermentation with a BASI polypeptide) which is less able to withstand hydrolytic cleavage of one or more of its peptide bonds over a specified time period and under specified conditions, including the pH and or temperature at which the cleavage resistance is tested. One result of proteolytic cleavage indicative that cleavage has occurred is the generation of smaller fragments (lower molecular weight) as compared to the molecular weight of the intact, non-cleaved parent molecule. An antibody or a functional fragment thereof disclosed herein comprising a hinge, a CH2 domain and a CH3 domain is “protease resistant” or “resistant to proteolysis” or has “increased resistance to proteolysis” when more than about 60%, 65%, 70%, 75%, 80%, 85%, 90%, 95%, 96%, 97%, 98%, 99% or 100% of a full length antibody remains intact for a given period of time (such as about 1, 2, 3, 4, 5, 6, 7, 8, 9, 10, 11, 12, 13, 14, 15, 16, 17, 18, 19, 20, 21, 22, 23, or 24 hours) when digested by a protease (such as, but not limited to, pepsin, matrix metalloprotease-3 (MMP-3), matrix metalloprotease-12 (MMP-12), pepsin, glutamyl endopeptidase V8 of Staphylococcus aureus (GluV8), immunoglobulin degrading enzyme of Streptococcus pyogenes (IdeS)), or expression host protease in a given buffer (e.g., Tris-buffered saline) at a given temperature (e.g., at 37 °C) at a given pH at a given antibody concentration with a given protease concentration (such as about approximately 1-2% (w/w) ratio to IgG). Amount of intact IgG can be assessed by SDS-PAGE.

### C. Modification of Antibodies

The antibodies or functional fragments thereof (such as antigen-binding fragments thereof) provided herein can be modified. Modifications of an antibody or antigen-binding fragment can improve one or more properties of the antibody, including, but not limited to, decreasing the immunogenicity of the antibody or antigen-binding fragment, improving the half-life of the antibody or antigen-binding fragment, such as reducing the susceptibility to proteolysis and/or reducing susceptibility to oxidation, and altering or improving of the binding properties of the antibody or antigen-binding fragment thereof. Exemplary modifications include, but are not limited to, modifications of the primary amino acid sequence of the antibody or functional fragment thereof and/or BASI polypeptide and alteration of the post-translational modification of the antibody or functional fragment thereof and/or BASI polypeptide.

Exemplary post-translational modifications include, for example, glycosylation, acetylation, pegylation, phosphorylation, amidation, derivatization with protecting/blocking group, proteolytic cleavage, linkage to a cellular ligand or other protein. Other exemplary modifications include attachment of one or more heterologous peptides to the antibody or functional fragment thereof and/or BASI polypeptide to alter or improve one or more properties of the antibody or antigen-binding fragment thereof.

### 1. Signal Sequences

In some embodiments, the antibodies disclosed herein can include a signal sequence. The signal sequence can be any signal sequence that facilitates protein secretion from a host cell (e.g., a filamentous fungal host cell). In particular embodiments, the antibody can comprise a signal sequence for a protein that is known to be highly secreted from a host cell in which the fusion protein is to be produced. The signal sequence employed can be endogenous or non-endogenous to the host cell in which the antibody or functional fragment thereof and/or BASI polypeptide is to be produced.

Suitable signal sequences are known in the art (see, e.g., Ward et al, Bio/Technology 1990 8:435-440; and Paloheimo et al, Applied and Environmental Microbiology 2003 69:7073-7082). Non-limiting examples of suitable signal sequences include those of cellobiohydrolase I, cellobiohydrolase II, endoglucanases I, II and III, α-amylase, aspartyl proteases, glucoamylase, phytase, mannanase, a and B glucosidases, bovine chymosin, human interferon and human tissue plasminogen activator and synthetic consensus eukaryotic signal sequences such as those described by Gwynne et al., (1987) Bio/Technology 5:713-719.

In some embodiments, if Trichoderma (e.g. T. reesei) is employed as a host cell, the signal sequence or carrier of T. reesei mannanase I (Man5A, or MANI), T. reesei cellobiohydrolase II (Cel6A or CBHII), endoglucanase I (Cel7b or EGI), endoglucanase II (Cel5a or EGII), endoglucanase III (Cel12A or EGIII), xylanases I or II (XynIIa or Xynllb) or T. reesei cellobiohydrolase I (Cel7a or CBHI) can be employed in the antibody or functional fragment thereof and/or BASI polypeptide.

In other embodiments, if an Aspergillus (e.g. A. niger) is employed as a host cell, the signal sequence or carrier of A. niger glucoamylase (GlaA) or alpha amylase can be employed in the fusion polypeptide. Aspergillus niger and Aspergillus awamori glucoamylases have identical amino acid sequences. Two forms of the enzyme are generally recognized in culture supernatants. GAI is the full-length form (amino acid residues 1-616) and GAII is a natural proteolytic fragment comprising amino acid residues 1-512. GAI is known to fold as two separate domains joined by an extended linker region. The two domains are the 471-residue catalytic domain (amino acids 1-471) and the 108 residue starch binding domain (amino acids 509-616), the linker region between the two domains being 36 residues (amino acids 472-508). GAII lacks the starch binding domain. Reference is made to Libby et al., (1994) Protein Engineering 7:1109-1114. In some embodiments, the glucoamylase which is used as a carrier protein and including a signal sequence will have greater than 95%, 96%, 97%, 98% and 99% sequence identity with a catalytic domain of an Aspergillus or Trichoderma glucoamylase. The term “catalytic domain” refers to a structural portion or region of the amino acid sequence of a protein which possess the catalytic activity of the protein.

### 2. Carriers

In particular embodiments, the signal sequence can comprise a “carrier” that contains the signal sequence at its N-terminus, where the carrier is at least an N-terminal portion of a protein that is efficiently secreted by a cell. In certain embodiments, the signal sequence and the carrier protein are obtained from the same gene. In some embodiments, the signal sequence and the carrier protein are obtained from different genes. In additional embodiments, the carrier protein can be cleaved from the antibody during secretion, yielding mature antibody free of carrier.

The carrier protein can include all or part of the mature sequence of a secreted polypeptide. In some embodiments, full length secreted polypeptides are used. However, functional portions of secreted polypeptides can be employed. As used herein “portion” of a secreted polypeptide or grammatical equivalents means a truncated secreted polypeptide that retains its ability to fold into a normal, albeit truncated, configuration.

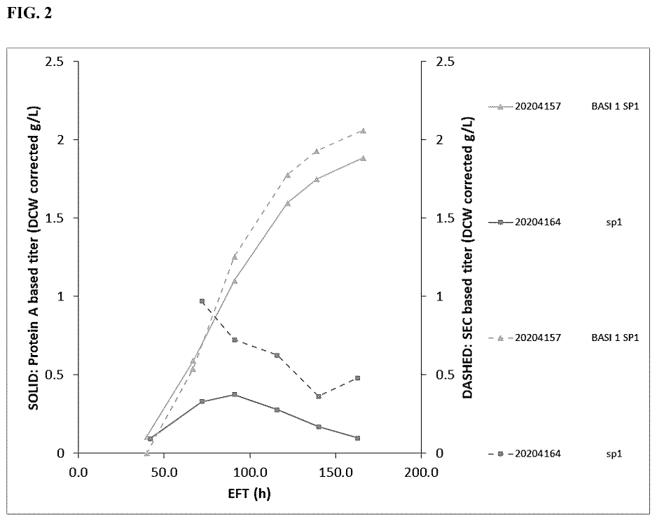

In some cases, the truncation of the secreted polypeptide means that the functional protein retains a biological function. In some embodiments, the catalytic domain of the secreted polypeptide is used, although other functional domains could be used, for example the substrate binding domain. In one embodiment, when glucoamylase is used as the carrier protein (e.g. glucoamylase from Aspergillus niger), functional portions retain the catalytic domain of the enzyme and include amino acids 1-471 (see, WO 03089614, e.g., Example 10, the disclosure of which is incorporated by reference herein). In another embodiment, when CBH I is used as the carrier protein (i.e. CBH I from Trichoderma reesei) functional portions retain the catalytic domain of the enzyme. Reference is made to SEQ ID NO:1 of FIG. 2 of WO 05093073, the disclosure of which is incorporated by reference herein, wherein the sequence encoding a Trichoderma reesei CBHI signal sequence, T. reesei CBH1 catalytic domain (also referred to as catalytic core or core domain) and T. reesei CBH1 linker is disclosed. In some embodiments, a CBH1 carrier protein and including a signal sequence will have greater than 95%, 96%, 97%, 98% and 99% sequence identity with SEQ ID NO: 1 of FIG. 2 of WO 05093073, the disclosure of which is incorporated by reference herein).

In general, if the carrier protein is a truncated protein, it is C-terminally truncated (i.e., contains an intact N-terminus). Alternatively, the carrier protein can be N-terminally truncated, or optionally truncated at both ends to leave a functional portion. Generally, such portions of a secreted protein which comprise a carrier protein comprise greater than 50%, greater than 70%, greater than 80% and greater than 90% of the secreted protein and, in some embodiments, the N-terminal portion of the secreted protein. In some embodiments, the carrier protein will include a linker region in addition to the catalytic domain. In some embodiments, a portion of the linker region of the CBHI protein can be used in the carrier protein.

In some embodiments, the first amino acid sequence comprising a signal sequence functional as a secretory sequence is encoded by a first DNA molecule. The second amino acid sequence comprising the carrier protein is encoded by a second DNA sequence. However, as described above the signal sequence and the carrier protein can be obtained from the same gene.

### 3. Antibody Conjugates and Derivatives

Any of the antibodies disclosed herein can include derivatives that are modified (i.e., by the covalent attachment of any type of molecule to the antibody). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. Further, antibodies can be conjugated to albumin in order to make the antibody or antibody fragment more stable in vivo or have a longer half-life in vivo. The techniques are well known in the art, see e.g., International Publication Nos. WO 93/15199, WO 93/15200, and WO 01/77137; and European Patent No. EP 413, 622. The present invention encompasses the use of antibodies or fragments thereof conjugated or fused to one or more moieties, including but not limited to, peptides, polypeptides, proteins, fusion proteins, nucleic acid molecules, small molecules, mimetic agents, synthetic drugs, inorganic molecules, and organic molecules.

The present invention encompasses the use of antibodies or fragments thereof recombinantly fused or chemically conjugated (including both covalent and non-covalent conjugations) to a heterologous protein or polypeptide (or fragment thereof, for example, to a polypeptide of at least 10, at least 20, at least 30, at least 40, at least 50, at least 60, at least 70, at least 80, at least 90 or at least 100 amino acids) to generate fusion proteins. The fusion does not necessarily need to be direct but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

The present invention further includes compositions comprising heterologous proteins, peptides or polypeptides fused or conjugated to antibody fragments. For example, the heterologous polypeptides may be fused or conjugated to a Fab fragment, Fd fragment, Fv fragment, F(ab)₂fragment, a VH domain, a VL domain, a VH CDR, a VL CDR, or fragment thereof Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Additional fusion proteins, e.g., of antibodies that specifically bind an antigen (e.g., supra), may be generated through the techniques of gene-shuffling, motif-shuffling, exon-shuffling, and/or codon-shuffling (collectively referred to as “DNA shuffling”). DNA shuffling may be employed to alter the activities of the antibody or functional fragment thereof disclosed herein (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2): 76-82; Hansson, et al., 1999, J. Mol. Biol. 287:265-76; and Lorenzo and Blasco, 1998, Biotechniques 24(2): 308-313. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to an Antigen may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, the antibodies disclosed herein, or analogs or derivatives thereof can be conjugated to a diagnostic or detectable agent. Such antibodies can be useful for monitoring or prognosing the development or progression of a cancer as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I) carbon (¹⁴C), sulfur (³⁵S), tritium (³H), indium ¹¹⁵In, ¹¹³In, ¹¹²In, ¹¹¹In), and and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸ Ge, ⁵⁷Co, ⁶⁵Zn, ⁸⁵Sr, ³²p, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, noradioactive paramagnetic metal ions, and molecules that are radiolabelled or conjugated to specific radioisotopes.

Use of the antibodies disclosed herein or fragments thereof conjugated to a therapeutic agent is also contemplated. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include ribonuclease, monomethylauristatin E and F, paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A more extensive list of therapeutic moieties can be found in PCT publications WO 03/075957, incorporated by reference herein.

Further, an antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

Techniques for conjugating therapeutic moieties to antibodies and related molecules are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119.

Methods for fusing or conjugating antibodies and related molecules to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337. The fusion of an antibody to a moiety does not necessarily need to be direct but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an antibody (such as any of those disclosed herein) should be chosen to achieve the desired prophylactic or therapeutic effect(s) for a particular disorder in a subject. A clinician or other medical personnel should consider the following when deciding on which therapeutic moiety or drug to conjugate to an antibody: the nature of the disease, the severity of the disease, and the condition of the subject.

### D. Polynucleotides

Another aspect of the compositions and methods disclosed herein is a polynucleotide or a nucleic acid sequence that encodes an antibody or functional fragment thereof or that encodes a BASI polypeptide for use in any of the methods disclosed herein.

A fusion DNA construct encoding an antibody or functional fragment thereof and/or a BASI polypeptide is provided herein, comprising in operable linkage a promoter; a first DNA molecule encoding a signal sequence; a second DNA molecule encoding a carrier protein; a third DNA molecule encoding an antibody (e.g. a heavy chain and/or a light chain) or functional fragment thereof; and a fourth DNA molecule encoding a BASI polypeptide. The components of the fusion DNA construct can occur in any order. Since the genetic code is known, the design and production of these nucleic acids is well within the skill of an ordinarily skilled artisan, given the description of the antibodies or functional fragments thereof and/or BASI polypeptide disclosed herein. In certain embodiments, the nucleic acids can be codon optimized for expression of the antibodies or functional fragments thereof and/or BASI polypeptide in a particular host cell. Since codon usage tables are available for many species of, for example, mammalian cells and filamentous fungi, the design and production of codon-optimized nucleic acids that encodes subject antibodies and/or BASI polypeptide would be well within the skill of one of skill in the art.

### E. Promoters

Examples of suitable promoters for directing the transcription of a nucleic acid in a host cell (for example, a filamentous fungal host cell) are promoters obtained from the genes for Aspergillus oryzae TAKA amylase, Rhizomucor miehei aspartic proteinase, Aspergillus niger neutral alpha-amylase, Aspergillus niger acid stable alpha-amylase (Korman et al (1990) Curr. Genet 17:203-212; Gines et al., (1989) Gene 79:107-117), Aspergillus niger or Aspergillus awamori glucoamylase (glaA) (Nunberg et al., (1984) Mol. Cell Biol. 4:2306-2315; Boel E. et al., (1984) EMBO J. 3:1581-1585), Rhizomucor miehei lipase, Aspergillus oryzae alkaline protease, Aspergillus oryzae triose phosphate isomerase, Aspergillus nidulans acetamidase (Hyner et al., (1983) Mol. Cell. Biol. 3:1430-1439), Fusarium venenatum amyloglucosidase, Fusarium oxysporum trypsin-like protease (WO 96/00787), Trichoderma reesei cellobiohydrolase I (Shoemaker et al. (1984) EPA EPO 0137280), Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof. Reference is also made to Yelton et al., (1984) Proc. Natl. Acad. Sci. USA 81:1470-1474; Mullaney et al., (1985) Mol. Gen. Genet. 199:37-45; Lockington et al., (1986) Gene 33:137-149; Macknight et al., (1986) Cell 46:143-147; Hynes et al., (1983) Mol. Cell Biol. 3:1430-1439. Higher eukaryotic promoters such as SV40 early promoter (Barclay et al (1983) Molecular and Cellular Biology 3:2117-2130) can also be useful. Promoters can be constitutive or inducible promoters. Exemplary promoters include a Trichoderma reesei cellobiohydrolase I or II, a Trichoderma reesei endoglucanase I, II or III, and a Trichoderma reesei xylanase II.

### F. Vectors

A polynucleotide encoding any of the antibodies or functional fragments thereof and/or BASI polypeptide disclosed herein can be present in a vector, for example, a phage, plasmid, viral, or retroviral vector. In certain embodiments, the vector can be an expression vector for expressing a subject fusion polypeptide in a filamentous fungal cell.

Vectors for expression of recombinant proteins are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.).

A fusion DNA construct can be constructed using well known techniques as is generally described for example in European Patent Application Publication No. 0 215 594, the disclosure of which is incorporated by reference herein.

Natural or synthetic polynucleotide fragments encoding for the polypeptide of interest (e.g. an immunoglobulin) can be incorporated into heterologous nucleic acid constructs or vectors, capable of introduction into and replication in a host cell (e.g., a filamentous fungal host cell).

Once a DNA construct or more specifically a fusion DNA construct is made it can be incorporated into any number of vectors as is known in the art. While the DNA construct will in some embodiments include a promoter sequence, in other embodiments the vector will include other regulatory sequences functional in the host to be transformed, such as ribosomal binding sites, transcription start and stop sequences, terminator sequences, polyadenylation signals, enhancers and or activators. In some embodiments, a polynucleotide encoding an antibody or functional fragment thereof and/or BASI polypeptide is inserted into a vector which comprises a promoter, signal sequence and carrier protein at an appropriate restriction endonuclease site by standard procedures. Such procedures and related sub-cloning procedures are deemed to be within the scope of knowledge of those skilled in the art.

Terminator sequences which are recognized by the expression host to terminate transcription can be operably linked to the 3′ end of the fusion DNA construct encoding the antibody or functional fragment thereof and/or BASI polypeptide to be expressed. Those of general skill in the art are well aware of various terminator sequences that can be used with host cells, such as, filamentous fungi. Non-limiting examples include the terminator from the Aspergillus nidulans trpC gene (Yelton M. et al., (1984) Proc. Natl. Acad. Sci. USA 81:1470-1474) or the terminator from the Aspergillus niger glucoamylase genes (Nunberg et al. (1984) Mol. Cell. Biol. 4:2306-2353) or the terminator from the Trichoderma reesei cellobiohydrolase I gene.

Polyadenylation sequences are DNA sequences which when transcribed are recognized by the expression host to add polyadenosine residues to transcribed mRNA. Examples include polyadenylation sequences from A. nidulans trpC gene (Yelton et al (1984) Proc. Natl. Acad. Sci. USA 81; 1470-1474); from A. niger glucoamylase gene (Nunberg et al. (1984) Mol. Cell. Biol. 4:2306-2315); the A. oryzae or A. niger alpha amylase gene and the Rhizomucor miehei carboxyl protease gene.

In further embodiments, the fusion DNA construct or the vector comprising the fusion DNA construct will contain a selectable marker gene to allow the selection of transformed host cells. Selection marker genes are well known in the art and will vary with the host cell used. Examples of selectable markers include but are not limited to ones that confer antimicrobial resistance (e.g. hygromycin, bleomycin, chloroamphenicol and phleomycin). Genes that confer metabolic advantage, such as nutritional selective markers can also find use. Some of these markers include amdS. Also, sequences encoding genes which complement an auxotrophic defect can be used as selection markers (e.g. pyr4 complementation of a pyr4 deficient A. nidulans, A. awamori or Trichoderma reesei and argB complementation of an argB deficient strain). Reference is made to Kelley et al., (1985) EMBO J. 4:475-479; Penttila et al., (1987) Gene 61:155-164 and Kinghorn et al (1992) Applied Molecular Genetics of Filamentous Fungi, Blackie Academic and Professional, Chapman and Hall, London, the disclosure of each of which are incorporated by reference herein.

### G. Host Cells

Provided herein are recombinant host cells comprising a heterologously expressed barley alpha-amylase subtilisin inhibitor (BASI) polypeptide; and a heterologously expressed monoclonal antibody or functional fragment thereof.

The expression cassette or vectors expressing a polynucleotide encoding a BASI polypeptide and/or a monoclonal antibody or functional fragment thereof described above can be introduced into a suitable expression host cell, which then expresses the corresponding polynucleotide encoding an antibody or functional fragment thereof and/or BASI polypeptide.

Suitable host cells include cells of any microorganism (e.g., cells of a bacterium, a protist, an alga, a fungus (e.g., a yeast or filamentous fungus), or other microbe), and can be cells of a bacterium, a yeast, a plant, or a filamentous fungus. Fungal expression hosts can be, for example, yeasts. Also suited are mammalian expression hosts such as mouse (e.g., NSO), Chinese Hamster Ovary (CHO), human embryonic kidney (HEK) or Baby Hamster Kidney (BHK) cell lines. Other eukaryotic hosts such as insect cells (such as Drosophila S2 cells) or viral expression systems (e.g., bacteriophages such as M13, T7 phage or Lambda, or viruses such as Baculovirus) are also suitable for producing the polypeptide.

Suitable host cells of the bacterial genera include, but are not limited to, cells of Escherichia, Proteus, Bacillus, Ralstonia, Lactobacillus, Lactococcus, Pseudomonas, Staphylococcus, and Streptomyces. Suitable cells of bacterial species include, but are not limited to, cells of Escherichia coli, Bacillus subtilis, Bacillus licheniformis, Bacillus megaterium, Lactobacillus brevis, Pseudomonas aeruginosa, Pseudomonas fluorescens, Pseudomonas stutzerei, Staphylococcus carnosus, Lactococcus lactis, Ralstonia eutropha, Proteus mirabilis, and Streptomyces lividans.

Suitable host cells of the genera of yeast include, but are not limited to, cells of Saccharomyces, Schizosaccharomyces, Candida, Hansenula, Pichia, Kluyveromyces, Yarrowia and Phaffia. Suitable cells of yeast species include, but are not limited to, cells of Saccharomyces cerevisiae, Schizosaccharomyces pombe, Candida albicans, Hansenula polymorpha, Yarrowia lipolytica, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Promoters and/or signal sequences associated with secreted proteins in a particular host of interest are candidates for use in the heterologous production and secretion of antibodies or functional fragments thereof and/or BASI polypeptide in that host or in other hosts. As a non-limiting example, in filamentous fungal systems, the promoters that drive the genes for cellobiohydrolase I (cbh1), glucoamylase A (glaA), TAKA-amylase (amyA), xylanase (exIA), the gpdA-promoter cbh1, cbhll, endoglucanase genes eg1-eg5, Cel61B, Cel74A, gpd promoter, Pgk1, pki1, EF-1alpha, tef1, cDNA1 and hex1 are suitable and can be derived from a number of different organisms (e.g., A. niger, T. reesei, A. oryzae, A. awamori, A. nidulans).

In some embodiments, the polynucleotide encoding an antibody or functional fragment thereof and/or BASI polypeptide is recombinantly associated with a polynucleotide encoding a suitable homologous or heterologous signal sequence that leads to secretion of the recombinant polypeptide into the extracellular (or periplasmic) space, thereby allowing direct detection in the cell supernatant (or periplasmic space or lysate). Suitable signal sequences for Escherichia coli, other gram-negative bacteria and other organisms known in the art include those that drive expression of the HlyA, DsbA, Pbp, PhoA, PelB, OmpA, OmpT or M13 phage Gill genes. For Bacillus subtilis, Gram-positive organisms and other organisms known in the art, suitable signal sequences further include those that drive expression of the AprE, NprB, Mpr, AmyA, AmyE, Blac, SacB, and for S. cerevisiae or other yeast, including the killer toxin, Bar1, Suc2, Mating factor alpha, Inu1A or Ggplp signal sequence. Signal sequences can be cleaved by a number of signal peptidases, thus removing them from the rest of the expressed protein.

In some embodiments, the antibody or functional fragment thereof and/or BASI polypeptide is/are expressed alone or as a fusion with additional peptides, tags or proteins located at the N- or C-terminus (e.g., 6XHis, HA or FLAG tags). Suitable fusions include tags, peptides or proteins that facilitate affinity purification or detection (e.g., 6XHis, HA, chitin binding protein, thioredoxin or FLAG tags), as well as those that facilitate expression, secretion or processing of the target beta-glucosidases. In addition to KEX2, further suitable processing sites include enterokinase, STE13, or other protease cleavage sites known in the art for cleavage in vivo or in vitro.

Polynucleotides encoding an antibody or functional fragment thereof and/or BASI polypeptide can be introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

### III. METHODS

### A. Methods for Producing Antibodies

Antibodies for use in the methods disclosed herein disclosed herein can be produced by any method known in the art for the synthesis of antibodies, in particular, by chemical synthesis or by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an antigen or immunogenic fragment thereof and once an immune response is detected, e.g., antibodies specific for the administered antigen are detected in the mouse serum, the mouse spleen is harvested and splenocytes isolated. The splenocytes are then fused by well-known techniques to any suitable myeloma cells, for example cells from cell line SP20 available from the ATCC. Additionally, a RIMMS (repetitive immunization, multiple sites) technique can be used to immunize an animal (Kilpatrick et al., 1997, Hybridoma 16:381-9). Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody wherein, the hybridoma may be generated by fusing splenocytes isolated from a mouse immunized with an antigen or immunogenic fragments thereof, with myeloma cells and then screening the hybridomas resulting from the fusion for hybridoma clones that secrete an antibody able to bind the administered antigen.

The antibodies for use in the methods disclosed herein can additionally contain novel amino acid residues in their hinge regions. Engineered antibodies can be generated by numerous methods well known to one skilled in the art. Non-limiting examples include, isolating antibody coding regions (e.g., from hybridoma) and introducing one or more hinge modifications of the invention into the isolated antibody coding region. Alternatively, the variable regions may be subcloned into a vector encoding comprising a modified hinge region (such as any of these disclosed herein). Additional methods and details are provided infra.

Antibody fragments that recognize specific an antigen can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies or functional fragments thereof disclosed herein can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an Antigen epitope of interest can be selected or identified with antigen, e.g., using labeled antigen or antigen bound or captured to a solid surface or bead. Examples of phage display methods that can be used to make the antibodies or functional fragments thereof disclosed herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in International Publication No. WO 92/22324; Mullinax et al., 1992, BioTechniques 12 (6): 864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma constant, and the PCR amplified VL domains can be cloned into vectors expressing a VL constant region, e.g., human kappa or lamba constant regions. It is contemplated that the constant region comprises a modified hinge (such as any of the modified hinges disclosed herein). In certain embodiments, the vectors for expressing the VH or VL domains comprise a promoter, a secretion signal, a cloning site for both the variable and constant domains, as well as a selection marker such as neomycin. The VH and VL domains may also be cloned into one vector expressing the desired constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816397, and 6,311,415.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT Publication Nos. WO 98/46645, WO 98/50433, WO 98/24893, WO98/16654, WO 96/34096, WO 96/33735, and WO 91/10741.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. In a specific embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, or greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28 (4/5): 489-498; Studnicka et al., 1994, Protein Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119-25 (2002), Caldas et al., Protein Eng. 13 (5): 353-60 (2000), Morea et al., Methods 20 (3): 267-79 (2000), Baca et al., J. Biol. Chem. 272 (16): 10678-84 (1997), Roguska et al., Protein Eng. 9 (10): 895-904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen or immunogenic fragments thereof. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598.

Further, the antibodies or functional fragments thereof disclosed herein can, in turn, be utilized to generate anti-idiotype antibodies that “mimic” a receptor using techniques well known to those skilled in the art. (See, e.g., Greenspan & Bona, 1989, FASEB J. 7 (5): 437-444; and Nissinoff, 1991, J. Immunol. 147 (8): 2429-2438). For example, antibodies of the invention which bind to and competitively inhibit the binding of a receptor (as determined by assays well known in the art and disclosed infra) to its ligands can be used to generate anti-idiotypes that “mimic” the ligand and, as a consequence, bind to and neutralize the receptor and/or its ligands. Such neutralizing anti-idiotypes or Fab fragments of such anti-idiotypes can be used in therapeutic regimens to neutralize a ligand and/or its receptor. Methods employing the use of polynucleotides comprising a nucleotide sequence encoding an antibody or a fragment thereof are provided herein.

In one embodiment, the nucleotide sequence encoding an antibody that specifically binds an antigen is obtained and used to generate the antibody or functional fragment thereof disclosed herein. The nucleotide sequence can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+RNA, isolated from any tissue or cells expressing the antibody, such as hybridoma cells selected to express an antibody) by PCR amplification using synthetic primers that hybridize to the 3′ and 5′ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999)), to generate antibodies having a different amino acid sequence by, for example, introducing deletions, and/or insertions into desired regions of the antibodies.

In a specific embodiment, one or more of the CDRs is inserted within framework regions using routine recombinant DNA techniques. The framework regions may be naturally occurring or consensus framework regions, including, but not limited to, human framework regions (see, e.g., Chothia et al., 1998, J. Mol. Biol. 278:457-479 for a listing of human framework regions). It is contemplated that the polynucleotide generated by the combination of the framework regions and CDRs encodes an antibody that specifically binds to an Antigen. In one embodiment, as discussed supra, one or more amino acid substitutions may be made within the framework regions, and, in certain embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

The hinge of antibodies identified from such screening methods can be modified as described supra to generate an antibody incorporating a modified hinge, such as any of those disclosed above. It is further contemplated that the antibodies disclosed herein are useful for the prevention, management and treatment of a disease, disorder, infection, including but not limited to inflammatory diseases, autoimmune diseases, bone metabolism related disorders, angiogenic related disorders, infection, and cancer. Such antibodies can be used in the methods and compositions disclosed herein.

### B. Recombinant Expression of Antibodies and BASI Polypeptides

Also provided herein are methods for decreasing proteolysis of a heterologously expressed monoclonal antibody or functional fragment thereof comprising culturing a recombinant cell comprising a heterologously expressed barley alpha-amylase subtilisin inhibitor (BASI) polypeptide; and the heterologously expressed monoclonal antibody or functional fragment thereof under suitable conditions for production of the heterologously expressed antibody or functional fragment thereof and BASI polypeptide.

Recombinant expression of antibodies or functional fragments thereof (as well as derivatives, analogs or fragments thereof) and BASI polypeptides, requires construction of an expression vector containing a polynucleotide that encodes the antibody or functional fragment thereof and the BASI polypeptide. Once a polynucleotide or polynucleotides encoding an antibody and/or BASI polypeptide has been obtained, the vector for the production of the antibody and/or BASI polypeptide can be produced by recombinant DNA technology using techniques well known in the art. Thus, methods for preparing a protein by expressing a polynucleotide containing an antibody-encoding nucleotide sequence and/or a BASI-encoding nucleotide sequence are described herein. Methods that are well known to those skilled in the art can be used to construct expression vectors containing antibody and BASI coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce an antibody and/or BASI polypeptide (such as any of those disclosed herein). In specific embodiments for the expression of antibodies comprising double-chained antibodies, vectors encoding both the heavy and light chains may be co-expressed in the host cell for expression of the entire immunoglobulin molecule.

In some embodiments, recombinant co-expression of a BASI polypeptide and a monoclonal antibody or functional fragment thereof results in decreased proteolysis of the monoclonal antibody or functional fragment thereof compared to recombinant expression of the monoclonal antibody or functional fragment thereof alone (i.e. without co-expression with a BASI polypeptide). In some embodiments, recombinant co-expression of a BASI polypeptide and a monoclonal antibody or functional fragment thereof results in between about 5%-100%, 10-90%, 20%-80%, 30%-70%, 40%-60%, 50%-100%, 50%-90%, 50%-75%, 60%-100%, 60%-90%, 60%-80%, 75%-100%, 75%-95%, 80%-100%, 80%-90%, such as any of about 5%, 10%, 15%, 20%, 25%, 30%, 35%, 40%, 45%, 50%, 55%, 60%, 65%, 70%, 75%, 80%, 85%, 90%, 95%, or 100% (inclusive of all values falling in between these percentages) decreased proteolysis of the monoclonal antibody or functional fragment thereof compared to recombinant expression of the monoclonal antibody or functional fragment thereof alone.

Further provided herein is a method for decreasing proteolysis of a heterologously expressed monoclonal antibody or functional fragment thereof comprising isolating or purifying the antibody or functional fragment thereof in the presence of an exogenously added barley alpha-amylase subtilisin inhibitor (BASI) polypeptide. In some embodiments, isolating or purifying the antibody or functional fragment thereof in the presence of an exogenously added BASI polypeptide results in decreased proteolysis of the monoclonal antibody or functional fragment thereof compared to isolation or purification of the monoclonal antibody or functional fragment in the absence of an exogenously added BASI polypeptide. In some embodiments, isolating or purifying the antibody or functional fragment thereof in the presence of an exogenously added BASI polypeptide results in between about 5%-100%, 10-90%, 20%-80%, 30%-70%, 40%-60%, 50%-100%, 50%-90%, 50%-75%, 60%-100%, 60%-90%, 60%-80%, 75%-100%, 75%-95%, 80%-100%, 80%-90%, such as any of about 5%, 10%, 15%, 20%, 25%, 30%, 35%, 40%, 45%, 50%, 55%, 60%, 65%, 70%, 75%, 80%, 85%, 90%, 95%, or 100% (inclusive of all values falling in between these percentages) decreased proteolysis of the monoclonal antibody or functional fragment thereof compared to isolation or purification of the monoclonal antibody or functional fragment in the absence of an exogenously added BASI polypeptide.

### C. Purification of Antibodies

Methods for purification of polypeptides, including the antibodies or antigen-binding fragments thereof provided herein, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

Proteins, such as the antibodies or antigen-binding fragments thereof provided herein, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His6 and affinity purified with myc antibody, glutathione resin and Ni-resin, respectively. Purity can be assessed by any method known in the art including gel electrophoresis and staining and spectrophotometric techniques.

Typically, antibodies and portions thereof are purified by any procedure known to one of skill in the art. The antibodies can be purified to substantial purity using standard protein purification techniques known in the art including but not limited to, SDS-PAGE, size fraction and size exclusion chromatography, ammonium sulfate precipitation, chelate chromatography, ionic exchange chromatography or column chromatography. For example, antibodies can be purified by column chromatography. Exemplary of a method to purify antibodies is by using column chromatography, wherein a solid support column material is linked to Protein G, a cell surface-associated protein from Streptococcus, that binds immunoglobulins with high affinity. The antibodies can be purified to 60%, 70%, 80% purity and typically at least 90%, 91%, 92%, 93%, 94%, 95%, 96%, 97%, 98% or 99% purity. Purity can be assessed by standard methods such as by SDS-PAGE and Coomassie staining.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides also can be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

The invention can be further understood by reference to the following examples, which are provided by way of illustration and are not meant to be limiting.

### EXAMPLES

### Example 1: Construction of Trichoderma reesei Strains for Co-Expression of BASI and Antibodies

pIT and pT1A vectors: Two plasmids, pI1 and pT1A, which are pUC18-based E. coli vectors, were used for the subcloning of fungal expression cassettes. Each plasmid contained two T. reesei QM6a DNA sequences (upstream and downsteam) of approximately 1 kb lengths, allowing for homologous recombination at the respective genomic loci. The pI1 vector was used for targeted integration at chr3 and has the native pyr2 as a selectable marker, while pT1A vector was used for targeted integration at chr2 and encodes amdS for selection on acetamide as a sole nitrogen source. Plasmid pI1 was constructed using Gibson assembly using the following four PCR products: (1) UHR (upstream homology region), amplified from QM6a genomic DNA (from chr3); (2) pyr2 gene (T. reesei orotate phosphoribosyl transferase) as described by Jørgensen, (2014), amplified from vector pTrex8gM (3) DHR (downstream homology region), amplified from QM6a genomic DNA (chr3); (4) pUC18 backbone (pUC18 origin of replication and E. coli b1a, encoding β-lactamase), amplified from vector pUC18. PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) cells and transformation was carried out according to standard protocol. Plasmid DNA was isolated from E. coli colonies using the NucleoSpin Plasmid Mini Kit (Macherey-Nagel, Düren, Germany) according to standard protocol. The pT1A plasmid similarly constructed with two exceptions, that the homologous upstream and downstream sequences target a genomic location of chr2, and the selection marker was the Aspergillus nidulans amdS gene (GenBank: BN001303.1).

pI1_X_Antibody C LC construct: Plasmid pI1_X_Antibody C LC plasmid was constructed using the GeneArt™ Seamless Cloning and Assembly Kit (Thermofisher, Waltham U.S.), assembling the following two PCR products: (1) the vector backbone amplified from the pI1 vector, which contains the homology region for targeted integration at chr3 and the native pyr2 as a selectable marker; (2) the antibody C light chain (LC) that was fused to the Aspergillus niger glucoamylase core and linker (GenBank: HQ537427.1) and flanked by the Aspergillus tubingensis xlnA promoter and native cbhl terminator amplified from synthetic DNAs synthesized by Twist Bioscience (San Francisco, U.S.). PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

pI1_C_Antibody C HC construct: Plasmid pI1_C_Antibody C HC plasmid was constructed using the GeneArt™ Seamless Cloning and Assembly Kit (Thermofisher, Waltham U.S.), assembling the following two PCR products: (1) the vector backbone amplified from the pI1 vector, which contains the homology region for targeted integration at chr3 and the native pyr2 as a selectable marker; (2) the antibody C heavy chain (HC) that was fused to the Trichoderma reesei CBH1 core and linker (XM_006969162.1) and flanked by the native cbhl promoter and terminator amplified from synthetic DNAs synthesized by Twist Bioscience (San Francisco, U.S.). PCR reactions were carried out using Q5 High-Fidelity DNA Polymerase (NEB—Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

pT1A_C_Antibody C HC construct: The pT1A_X_Antibody C HC plasmid was constructed using the GeneArt™ Seamless Cloning and Assembly Kit (Thermofisher, Waltham U.S.), assembling the following three PCR products: (1) the vector backbone PCR amplified from the pT1A vector which contains the homology region for targeted integration at ch2; (2) the Aspergillus nidulans amdS marker (GenBank: BN001303.1); (3) the antibody C heavy chain (HC) that was fused to the Trichoderma reesei CBH1 core and linker (XM_006969162.1) and flanked by the native cbh1 promoter and terminator amplified from synthetic DNAs synthesized by Twist Bioscience (San Francisco, U.S.). PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

pTIA_accessory protein_X_C_Antibody C HC construct: Plasmid pTIA_accessory protein_X_X_Antibody C HC construct was constructed using the GeneArt™ Seamless Cloning and Assembly Kit (Thermofisher, Waltham U.S.), assembling the following four PCR products: (1) the vector backbone amplified from the pT1A vector which contains the homology region for targeted integration at ch2; (2) the Aspergillus nidulans amdS marker (GenBank: BN001303.1); (3) an overexpression cassette for a native Trichoderma reesei protein; (4) the antibody C heavy chain (HC) that was fused to the Trichoderma reesei CBH1 core and linker (XM_006969162.1) and flanked by the native cbhl promoter and terminator amplified from synthetic DNAs synthesized by Twist Bioscience (San Francisco, U.S.). PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Transformation of Antibody C: Donor DNA amplified from the pI1_cHC expression vectors used for the transformation reactions contained approximately 1.0 kb upstream homology region for targeting at chr3, the expression cassette, and the partial fragment of the pyr2 marker. The expression cassette comprised of the heavy chain (HC) that was fused to the native cbhl core and linker (XM_006969162.1) and flanked by the native cbhl promoter and terminator. The donor DNA amplified from the pI1_X_xLC expression vectors used for the transformation reaction contained approximately 1.0 kb downstream homology region, the expression cassette, and the partial fragment of the pyr2 marker. The expression cassette comprised of the light chain (LC) that was fused to the Aspergillus niger glucoamylase core and linker (GenBank: HQ537427.1) and flanked by the Aspergillus tubingensis xlnA promoter and native cbhl terminator.

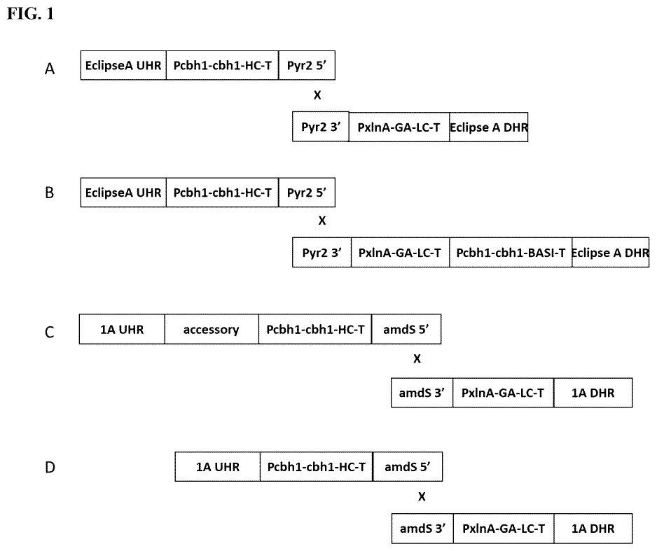

Antibody A and B constructs: Four different constructs based on “pI1” and “pTIA” backbones were constructed with expression cassettes for both Antibody A and Antibody B sequences for expression in Trichoderma reesei. This enabled the targeting of 1 copy of each antibody chain to the two respective native loci for integration at loci on chr2 and chr3. For the p1A constructs the Aspergillus nidulans amdS marker was used for the selection. DNAs encoding antibody peptides were synthesized by Twist Bioscience (San Francisco, U.S.) These synthetic heavy chain and light chain fragments were cloned into the pI1 and p1A vectors resulting in: pI1_cHC_AntibodyA, pI1_cHC_AntibodyB, pI1_X_xLC_AntibodyA, pI1_X_xLC_AntibodyB, p1A_cHC_AntibodyA, p1A_cHC_AntibodyB, p1A_X_xLC_AntibodyA, and p1A_X_xLC_AntibodyB. Donor DNA amplified from the pI1_cHC expression vectors used for the transformation reactions with primer pair RAS210 and JC831 contained approximately 1.0 kb upstream homology region, the expression cassette, and the partial fragment of the pyr2 marker. The expression cassette comprised of the heavy chain (HC) that was fused to the native cbhl core and linker (XM_006969162.1) and flanked by the native cbhl promoter and terminator. The donor DNA amplified from the pI1_X_xLC expression vectors used for the transformation reaction RAS500 and RAS213 contained approximately 1.0 kb downstream homology region, the expression cassette, and the partial fragment of the pyr2 marker. The expression cassette comprised of the either the light chain (LC) that was fused to the Aspergillus niger glucoamylase core and linker (GenBank: HQ537427.1) and flanked by the Aspergillus tubingensis xlnA promoter and native cbhl terminator (FIG. 1).

Donor DNA amplified from the p1A_cHC expression vectors used for the transformation reactions with primer pair SK4242 and SK4244 contained approximately 1.0 kb upstream homology region for targeting at chr3, the expression cassette, and the partial fragment of the amdS marker. The expression cassette comprised of either the Antibody A or Antibody B heavy chain (HC) that was fused to the native cbhl core and linker (XM_006969162.1) and flanked by the native cbhl promoter and terminator. The donor DNA amplified from the p1A_X_xLC expression vectors used for the transformation reaction with primer pair RAS680 and RAS693 contained the approximately 1.0 kb downstream homology region, the expression cassette, and the partial fragment of the amdS marker. The expression cassette comprised the light chain (LC) that was fused to the Aspergillus niger glucoamylase core and linker (GenBank: HQ537427.1) and flanked by the Aspergillus tubingensis xlnA promoter and native cbhl terminator. PCR reactions were carried out using Q5 High-Fidelity DNA Polymerase (NEB-Ipswich, U.S.) according to standard protocol. This split marker strategy increases the probability of single copy integration at the targeted loci.

LC-BASI construct: To test the effect of BASI co-expression on proteolysis of antibody, a fusion BASI expression cassette was incorporated into the pI1_X_xLC_Antibody A and pI1_X_xLC_Antibody B vectors. The BASI expression cassette comprised the native Trichoderma reesei cbh2 (GenBank: M55080.1) promoter and the Aspergillus nidulans trpC (GenBank: Z32524.1) terminator regulating BASI expression. The synthetic BASI mature sequence was synthesized by Twist Bioscience (South San Francisco, U.S.) This mature sequence was fused to the native Cbh1 core and linker (XM_006969162.1) resulting in a fusion chimera, analogous to the heavy chain and light chain fusion constructs. This Cbh1_BASI fusion expression cassette comprising of the cbh2 promoter, cbhl core and linker, BASI mature sequence, and the trpC terminator was cloned into both the pI1_X_xLC_Antibody A and pI1_X_xLC_Antibody B plasmids. These BASI constructs were made using The Q5® High-Fidelity DNA Polymerase (NEB-Ipswich, U.S.), GeneArt Seamless Cloning and Assembly Kit (Thermofisher, Waltham U.S.), Qiagen DNA purification kits (Hilden, Germany), primers from Integrated DNA Technologies (Coralville, U.S.), using standard methods known to one skilled in the art. The resulting constructs were referred to as pI1_X_xLC_Antibody A_BASI fusion and pI1_X_xLC_Antibody B_BASI fusion. Donor DNA for used transformation was generated by PCR. This donor DNA fragment contained a partial fragment of the pyr2 marker, light chain expression cassette as described previously, BASI fusion expression cassette, and approximately 1.0 kb downstream homology region to chromosome 3. Donor DNA fragments of the heavy chain expression cassettes and the light chain expression cassettes, with and without the additional BASI expression cassette were introduced into Trichoderma reesei strain T4_X.

pAS0025: Plasmid pAS0025 was constructed using the GeneArt™ Seamless Cloning and Assembly Kit (Thermofisher, Waltham U.S.) assembling the following two products: (1) The vector backbone containing Trichoderma reesei CBH1 core flanked by CBH1 promoter, CBH1 terminator, and T. reesei pyr2 marker and was cut with restriction enzymes BamHI and Xmal by standard protocol; (2) ccdB gene and chloramphenicol resistance marker (Invitrogen) synthetic DNA (IDT, Coralville, IA). The assembled product was added to 50 μL One Shot™ ccdB Survival Cells Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

pJC110: Plasmid pJC110 was constructed using the GeneArt™ Seamless Cloning and Assembly Kit (Thermofisher, Waltham U.S.) assembling the following two products: (1) The vector backbone Aspergillus nidulans amdS marker (GenBank: BN001303.1) and cut with restriction enzyme AvrII directly downstream of this marker according to standard protocol; (2) Expression cassette with Trichoderma reesei CBH1 promoter and core, PaeI restriction site, ccdB gene and chloramphenicol resistance marker (Invitrogen), and Trichoderma reesei terminator amplified from vector pAS0025. The ccdB gene and chloramphenicol resistance marker requires a specific E. coli strain to survive and reduces the amount of background during screening as described by Invitrogen. PCR reactions were carried out using Q5 High-Fidelity DNA Polymerase (NEB-Ipswich, U.S.) according to standard protocol. The assembled product was added to 50 μL One Shot™ ccdB Survival Cells Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Antibody D LC construct (p.JC160): Plasmid pJC160 was constructed using the GeneArt™ Seamless Cloning and Assembly Kit (Thermofisher, Waltham U.S.) assembling the following two products: (1) The vector backbone pJC110 containing expression cassette with Trichoderma reesei CBH1 promoter, core, terminator, and Aspergillus nidulans amdS marker (GenBank: BN001303.1) that was cut with restriction enzymes PaeI and AvrII according to standard protocol; (2) Antibody D light chain flanked by partial Trichoderma reesei CBH1 core and Trichoderma reesei CBH1 terminator amplified from synthetic DNAs synthesized by Twist Bioscience (San Francisco, U.S.). PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Pegl1-accessory protein_X-Antibody D_LC construct (pSK736): Plasmid pSK736 was constructed using the GeneArt™ Seamless Cloning and Assembly Kit (Thermofisher, Waltham U.S.), assembling the following three PCR products: (1) the vector backbone PCR amplified from pCR TOPO Blunt II (Invitrogen); (2) the accessory protein_X expression cassette containing Trichoderma reesei promoter egll amplified from Trichoderma strain RL-P37 and Trichoderma reesei accessory protein_X CDS and terminator amplified from Trichoderma strain RL-P37 and fused together by fusion PCR; (3) the Antibody D LC expression cassette contains Antibody D LC and fused to the Trichoderma reesei CBH1 core and linker (XM_006969162.1) and flanked by the native cbhl promoter and terminator from Trichoderma strain RL-P37 along with the native Aspergillus nidulans amdS marker (GenBank: BN001303.1) amplified from vector pJC160. PCR reactions were carried out using Q5 High-Fidelity DNA Polymerase (NEB—Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Pcbh2-accessory protein_X-Antibody D LC construct (p.JC173): Plasmid pJC173 was constructed using the GeneArt™ Seamless Cloning and Assembly Kit (Thermofisher, Waltham U.S.), assembling the following two PCR products: (1) the vector backbone amplified from pSK736, which contains Trichoderma reesei accessory protein_X and the Antibody D LC expression cassette that was fused to the Trichoderma reesei CBH1 core and linker (XM_006969162.1) and flanked by the native cbhl promoter and terminator along with the Aspergillus nidulans amdS marker (GenBank: BN001303.1); (2) the Trichoderma reesei CBH2 promoter amplified from genomic DNA from Trichoderma reesei strain RL-P37. PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Antibody D_HC construct (p.JC158): Plasmid pJC158 was constructed using the GeneArt™ Seamless Cloning and Assembly Kit (Thermofisher, Waltham U.S.) assembling the following two products: (1) The vector backbone pAS0025 containing expression cassette with Trichoderma reesei CBH1 promoter, core, terminator, and pyr2 marker that is cut with restriction enzymes PaeI and AvrII according to standard protocol; (2) Antibody D HC flanked by partial Trichoderma reesei CBH1 core and Trichoderma reesei CBH1 terminator amplified from synthetic DNAs synthesized by Twist Bioscience (San Francisco, U.S.). PCR reactions were carried out using Q5 High-Fidelity DNA Polymerase (NEB-Ipswich, U.S.) according to standard protocol. The assembled product was added to 50 μL TOP10 Chemically Competent E. coli (Thermofisher, Waltham U.S.) and transformation was carried out according to standard protocol. Plasmid DNA was isolated from E. coli colonies using the Qiaquick Spin Mini Prep DNA kit (Hilden, Germany) according to standard protocol.

Generating Donor DNAs for transformation of Antibody D: The donor DNA was generated from vector pJC173 via PCR amplification. The PCR fragment comprised of the Trichoderma reesei accessory protein_X, driven by Trichoderma reesei cbh2 promoter and the Antibody D LC expression cassette containing the Antibody D LC fused to Trichoderma reesei Cbh1 core and linker (XM_006969162.1) and flanked by the native cbhl promoter and terminator sequences. The selection marker for the pJC173 donor fragment was the Aspergillus nidulans amdS gene (GenBank: BN001303.1). The donor DNA of the vector pJC158 comprised of Antibody D HC fused to the Trichoderma reesei CBH1 core and linker (XM_006969162.1), which was flanked by the native cbhl promoter and terminator. The selection marker for the expression cassette was the Trichoderma reesei pyr2 gene. PCR reactions were carried out using Q5 High-Fidelity DNA Polymerase (NEB-Ipswich, U.S.) according to standard protocol.

Transformation of Antibody D: The host T. reesei strain used for transformation is deleted for major cellulases and xylanases. The strain was transformed using a standard PEG-protoplast transformation method. Transformation mixtures containing approximately 5 μg of each DNA and 5×10⁶ protoplasts in a total volume of 250 μl were treated with 2 mL of 25% PEG solution, diluted with 2 volumes of 1.2M sorbitol/10 mM Tris, pH7.5/10 mM CaCl₂ solution, and mixed with 26 mL of 2% low melting agarose containing 1M sorbitol and acetamide as a sole nitrogen source, in minimal medium and distributed over 4 10 cm petri plates pre-poured containing 1.5% agarose, 1M sorbitol and acetamide in minimal media. Individual colonies were picked onto fresh 10 cm petri plates containing 1.5% agar, 1 g/L uridine, and acetamide. Once stable transformants were well sporulated, spores were harvested and used for inoculation of liquid cultures.

The T. reesei parental strain, T4_X described in the instant example was derived from T. reesei strain RL-P37 (NRRL Deposit No. 15709), as described by Sheir-Neiss and Montenecourt (1984, incorporated by reference herein). A strain, herein named “T4_X”, is a mutagenized derivative of RL-P37, with notable mutations in the pyr2 gene such that the strain requires uridine for growth and a nik1 (M743T) mutation that increases total protein production (see, e.g., U.S. Patent Application Publication No. 2018/0037919, incorporated by reference herein).

### Example 2: Production of BASI in Bacillus subtilis

This example demonstrates the production of BASI protein in liquid fermentation of B. subtilis. The inoculum was grown in a seed flask containing LB medium.

The production medium used to produce the BASI protein contained minerals, one or more carbon sources, and a complex nitrogen source. The BASI protein accumulated in the broth/cells.

Bacillus subtilis at 14L scale in liquid culture: Growth in 14 L fermentors consisted of 2 steps: generating the seed culture and generating the production culture while producing protein. Seed cultures were started by inoculating 30 mL of LB media into a 350 mL flask. The seed cultures were incubated at 180 rpm and 37 °C for roughly 2 h-until it was turbid. A volume of 30 mL of the seed culture was inoculated into each tank to bring the final volume to 7 kg of appropriate production medium. The production culture had 1-sided pH control (base addition only) during the experiment and was controlled at pH 7.1 with NH₄OH for base addition. The feed was triggered when the OUR reach 25 mmol/L/h and ramped from 0.28 g/min to 1.65 g/min over 10 hours.

Various parameters were monitored during the run and include, but are not limited to: CER (carbon dioxide evolution rate), OUR (oxygen uptake rate), pH, DO (dissolved oxygen), OD (optical density), etc.

### Example 3: Production of Monoclonal Antibody in Trichoderma reesei

This example demonstrates the production of a monoclonal antibody in liquid fermentation. Sufficient biomass was first generated and then the biomass was induced to produce the monoclonal antibody. The inoculum was grown in a seed flask containing citrate minimal medium and trace elements.

The production medium used to produce the monoclonal antibody contained minerals, one or more carbon sources, and optionally a complex nitrogen source.

The type and amount of complex nitrogen is show in Table 1. All runs used cottonseed flour if a complex nitrogen source was used.

TABLE 1
Trichoderma reesei run conditions

| Run | | Complex | | | Qs | MgSO4 | |
|---|---|---|---|---|---|---|---|
| Number | Scale | N (g/kg) | pH_{f} | T_{f} | (g/g/h) | (g/kg) | Notes |
| 20200442 | 14 L | 0 | 5.5 | 24 | 0.03 | 1 | |
| 20200482 | 14 L | 45 CSF* | 5.5 | 24 | 0.04 | 1 | |
| 20200483 | 14 L | 0 CSF | 5.5 | 24 | 0.04 | 1 | |
| 20204157 | benchtop | 45 CSF* | 5.5 | 24 | 0.04 | 1 | |
| 20204164 | benchtop | 45 CSF | 5.5 | 24 | 0.04 | 1 | |
| 20204179 | benchtop | 45 CSF | 5.5 | 24 | 0.04 | 1 | |
| 20204180 | benchtop | 45 CSF | 5.5 | 24 | 0.04 | 1 | BASI |
| | | | | | | | added |
| 20204181 | benchtop | 45 CSF | 6.5 | 24 | 0.04 | 1 | |
| 20204182 | benchtop | 45 CSF | 6.5 | 24 | 0.04 | 1 | BASI |
| | | | | | | | added |
| 20200576 | 14 L | 45 CSF | 5.5 | 24 | 0.04 | 1 | |
| 20200577 | 14 L | 45 CSF | 5.5 | 24 | 0.04 | 1 | |
| 20208053 | 500 L | 25 CSF | 7.0 | 20 | 0.04 | 0.7 | |
| 20200874 | 14 L | 25 CSF | 6.5 | 24 | 0.04 | 0.7 | |

• *CSF = cotton seed flour

Growth of Trichoderma reesei in Fed-Batch: Growth in fermenters consisted of 2 steps: generating the seed culture and generating the production culture. The seed culture is necessary to build up biomass for the production culture. Seed cultures were started by inoculating 50 mL of citrate minimal media with the appropriate strain. The seed cultures were incubated at 100-300 rpm and 28 °C for 2 days. A volume of 50 mL of the seed culture was inoculated into each tank containing production media. The production cultures were grown at 34 °C and had the temperature lowered to Tf at feed start. Various parameters were monitored during the runs and include but were not limited to: CER (carbon dioxide evolution rate), OUR (oxygen uptake rate), pH, DO (dissolved oxygen), DCW (dry cell weight), antibody production, and total protein (TP) production. Run 20204180 and 20204182 had BASI added to the run. BASI was added at the beginning of the run and again when production started (pH was raised, temperature was lowered, and feed was started). A sample was taken just prior to the second addition and analysis showed no BASI was present.

FIG. 2 shows there was a positive effect on the titer of the 150 kDa antibody species (size exclusion chromatography (SEC), right axis) of having BASI co-expressed in this strain. The protein A based titer and SEC based titer for the BASI negative strain appeared to reach a maximum early and then drop as the run progresses.

### Example 4: Fermentation of Strain Co-Expressing Antibody B and BASI

Strains t-BBD55, expressing Antibody B and BASI in run 20204157, and t-BBH66, expressing Antibody B in run 20204164, were fermented under the same conditions. Antibody titer was determined using two methods. Protein A HPLC reported on total antibody-related species, including degraded ones. Size Exclusion Chromatography (SEC) reported on the concentration of proteins separated by size. In this case, the peak eluting at a time consistent with mass ~150 kDa was quantitated. This will report only on intact antibodies, excluding antibody fragments and host proteins of masses significantly greater or less than 150 kDa.

The method of separating the various species of the fermentations, including the fusion protein, free CBH1, light and heavy chains, the minor degradation products and the full-length, mature, 150kd antibody molecule was accomplished by size exclusion chromatography. This mode of chromatography separates molecules by their size rather than molar mass.

The method employed an SRT-SEC column, 7.8 mm×300 mm long, at 300A (Sepax, Inc). The buffer (150 mM sodium phosphate and 150 mM sodium chloride, pH 6.8) was run under isocratic conditions. It was run at 1 ml/min at 25 °C for 20 minutes and protein detected by UV absorption at 222 nm in comparison to a CHO-expressed antibody standard.

Total antibody-species concentration was determined using a MacPac Protein A column (4×35 mm, ThermoFisher) on Agilent HPLC system. Sample supernatant was diluted 16× or 64× with Eluant A-PBS at pH 7.5. pH7 in an Agilent HPLC plate. 20 μl of this was loaded onto the column using a method running 100% Eluent A (50 mM Sodium Phosphate, 150 mM NaCl, pH 7.5) for 0.2 minutes, followed by running 100% eluent B (50 mM Sodium Phosphate, 150 mM NaCl, pH 2.5) for 0.8 minutes and then 100% Eluent A for 1.2 minutes, with detection at 220 nm. The antibody peak area was recorded. Based on the standard curve of purified antibody, peak area was converted to antibody concentration as milligram per milliliter. Supernatant concentrations were corrected for cell mass by adjusting as such “DCW corrected=Supernatant Conc*(1-DCW*4/1000)” where “DCW” is the dry cell weight measured in g/kg.

### Example 5: Stabilization of Antibodies in Trichoderma Broth by Supplementation with Exogenously Produced BASI

This Example demonstrates exogenous BASI stabilization of monoclonal antibodies produced in T. reesei.

### Stabilization of Antibody D in Harvested Broth Using BASI Filtrate

Four 50 mL tubes were filled with fermentation broth (Broth) expressing Antibody D (defined media), BASI filtrate, 1M Bis-Tris buffer and water as shown in Table 2.

TABLE 2
Experimental conditions

| | | Test ID | | | |
|---|---|---|---|---|---|
| | | 1 | 2 | 3 | 4 |
| BASI Dose | gBASI Filtrate/g-Broth | 0 | 0.5 | 1 | 3 |
| Broth | g | 6.1 | 6.1 | 6.1 | 6.2 |
| BASI Filtrate | g | 0.0 | 3.0 | 6.1 | 18.1 |
| MQ Water | g | 17.9 | 14.9 | 12.0 | 0.0 |
| 1M Bis Tris pH 6.5 | g | 1.2 | 1.2 | 1.2 | 1.2 |

The tubes were mixed well by inversion. 5 mL of each treated broth was transferred to a 15 mL tube followed by storage at 5 °C. SDS-PAGE analysis (Coomassie blue staining) was performed on days 7 and 28.

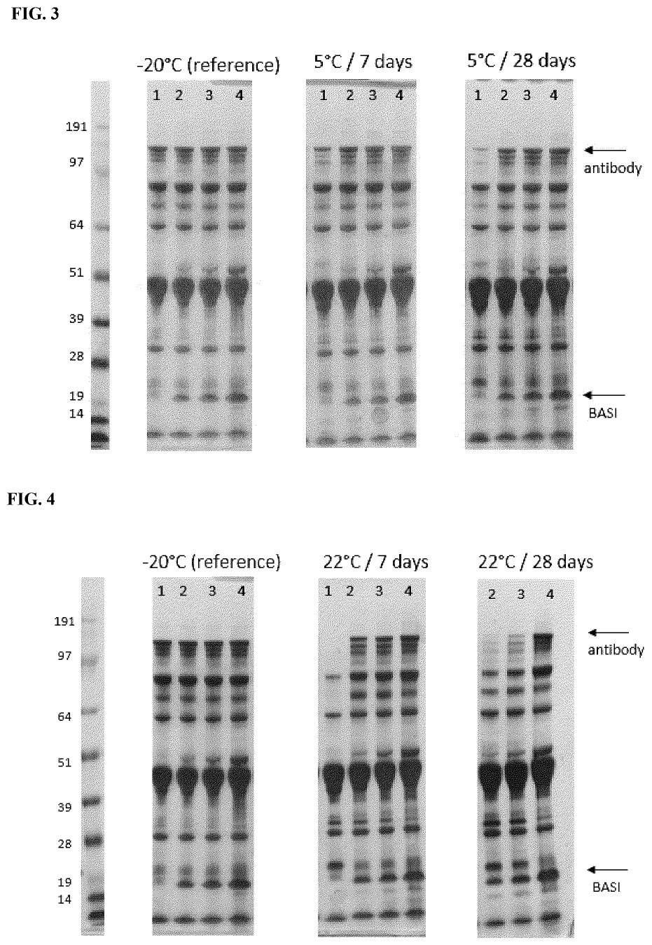

As shown in FIG. 3, untreated broth (experimental condition 1) had lost about 60% of antibody band by 7 days, and only trace amount remained after 28 days. Treated with BASI, antibody bands remained after 28 days storage at 5 °C for all levels (experimental conditions 2-4), with no significant loss compared to reference stored at −20 °C.

### Stabilization of Antibody D in Clarified Broth Using BASI Filtrate

Each of the treated broths used in the experiment described immediately above was centrifuged at 30,000 rpm for 20 min. The supernatant was collected from each tube and each filtered using a Steriflip filter unit. Each supernatant was then divided into two separate tubes with one set of tubes stored at −20 °C. (as reference) and one set at 22 °C.

As shown in FIG. 4, after 7 days at 22 °C, the antibody had complete loss in the non-treated clarified broth, while remaining in the BASI-treated clarified broth. No loss in antibody band at 10 g BASI filtrate per g broth was observed after 28 days storage at 22 °C.

### Stabilization of Antibody C in Clarified Broths Using BASI Filtrate

Four 14 mL tubes were filled with fermentation broth (Broth) expressing Antibody C in both complex (20200482: B2 2 copy Antibody C no endoT t-BAV65) and defined (20200483: B2 2 copy Antibody C no endoT; t-BAV65) media, BASI filtrate, 1M Bis-Tris buffer and water as shown in Table 3.

TABLE 3
Experimental conditions

| | | Treatment | | | |
|---|---|---|---|---|---|
| | | None | BASI | None | BASI |
| Broth Media | | complex | complex | defined | defined |
| Broth | g | 6.1 | 6.2 | 6.1 | 6.1 |
| BASI Filtrate | g | 0 | 6.1 | 0 | 6.3 |
| MQ Water | g | 16.9 | 11.1 | 16.8 | 10.9 |
| 1M pH 6.5 BisTris St [symbol] | g | 1.3 | 1.2 | 1.3 | 1.3 |

[symbol]

indicates data missing or illegible when filed

Each treated broth was centrifuged at 30,000 rpm for 20 min. Supernatants were collected and sterile filtered using a Steriflip filter unit. Each sample was divided into three tubes. One set of tubes was stored at −20 °C, one set at 5 °C and one set at 22 °C. SDS-PAGE analysis (Coomassie blue staining) was performed on day 4.

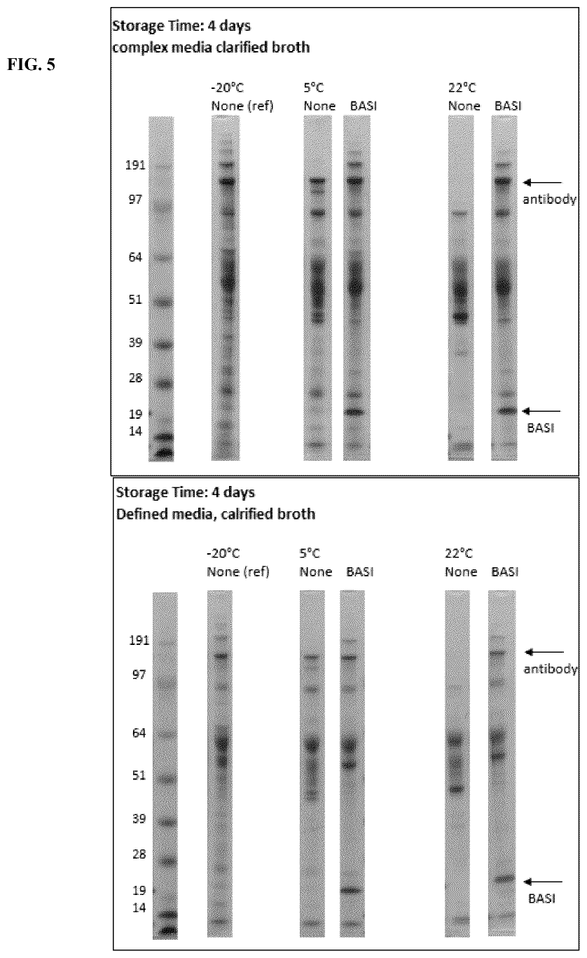

As shown in FIG. 5, after 4 days, untreated clarified broth exhibited a complete loss of antibody bands when stored at 22 °C. In contrast, with BASI addition, the antibody band remained at level comparable to the −20 °C reference sample.

### Stabilization of Antibody D in UF Concentrate Using BASI Filtrate

Ultralfiltered (UF) concentrate derived from the fermentation broth expressing Antibody D (defined media) as described above was prepared for a storage stability study as shown in Table 4.

TABLE 4
Experimental conditions

| | | None | 1 | 2 |
|---|---|---|---|---|
| UF Conc | g | 20 | 10.1 | 10 |
| Water | g | 16 | 15.6 | 0 |
| 1M BisTris, pH 6.5 | g | 4 | 3.5 | 3.4 |
| BASI Filtrate | g | 0 | 5 | 20.1 |

Each solution was mixed via inversion and sterile filtered using a Steriflip filter unit. Each sample was divided into three tubes with one set stored at −20 °C, one set at 5 °C and one set at 22 °C. SDS-PAGE analysis (Coomassie blue staining) was performed on day 5.

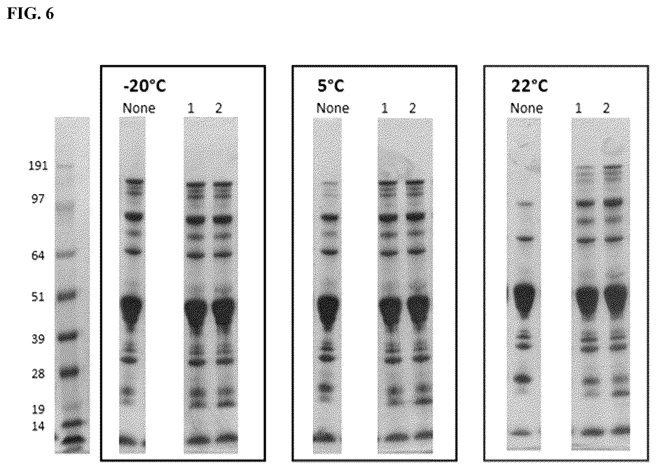

As shown in FIG. 6, after 5 days, UF concentrate without BASI stored at 5 °C had lost most of the antibody band. With BASI addition, however, the antibody band for both doses were similar to the corresponding −20 °C reference. With 22 °C storage, UF concentrate without BASI treatment exhibited complete loss of antibody, while antibody bands were still visible in UF concentrate treated with BASI.

### Stabilization of Antibody D in UF Concentrate Using Purified BASI

Ultralfiltered (UF) concentrate derived from the fermentation broth expressing Antibody D (defined media) as described above was prepared along with a purified BASI solution prepared from BASI shake flask filtrate as described above. Specifically, the purified BASI solution was prepared using a HiTrap SP XL 5 mL prepacked column. The column was equilibrated using a 50 mM sodium acetate equilibration buffer at pH 5. Elution was performed with same buffer from no salt to 0.5M NaCl over 10CV. Solutions for a storage stability study were prepared as shown in Table 5.

TABLE 5
Experimental conditions

| | | ID | | | |
|---|---|---|---|---|---|
| | | None | M | L | H |
| BASI Dose | g/kg-UFC | 0 | 1 | 0.25 | 1.9 |
| UFC | g | 5.1 | 5.3 | 5.1 | 5.0 |
| BASI, purified solution | g | 0.0 | 4.7 | 1.1 | 8.9 |
| Water | g | 8.5 | 4.0 | 7.4 | 0.0 |
| 1M BisTris, pH 6.5 | g | 1.6 | 1.6 | 1.6 | 1.6 |

Each solution was mixed via inversion and sterile filtered using a Steriflip filter unit. Each sample was divided into three tubes with one set stored at −20 °C, one set at 5 °C and one set at 22 °C. SDS-PAGE analysis (Coomassie blue staining) was performed on day 2, 4, and 10.

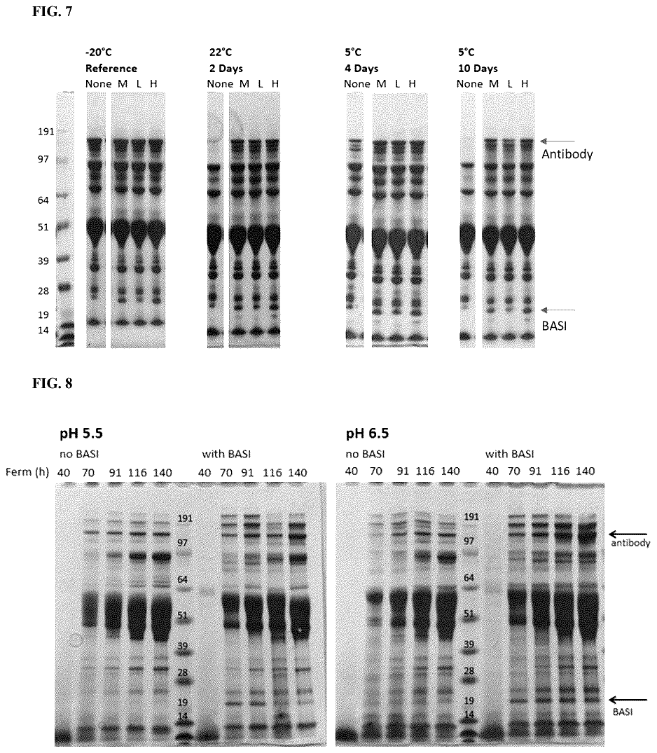

As shown in FIG. 7, without BASI addition, complete loss of antibody band was observed by day 2 at 22 °C and between day 4 and 10 at 5 °C. BASI addition stabilized the antibody with all samples have antibody bands retained.

### Stabilization of Antibody a During Fermentation Using BASI UFC

Complex fermentation media (with cotton seed flour) expressing Antibody A along with sterile filtered BASI concentrate was prepared as described above. Sterile filtered BASI UF concentrate (UFC) was added to each fermentation tank as shown in Table 6.

TABLE 6
Fermentation conditions

Tank

| | 1 | 2 | 3 | 4 |
|---|---|---|---|---|
| Fermentation pH | 5.5 | 5.5 | 6.5 | 6.5 |
| BASI addition (g BASI UFC) | | | | |
| Fermentation start | None | 20 | None | 20 |
| Feed Start | None | 10 | None | 10 |

Samples were collected at 40, 70, 91, 116, and 140 hours over the course of the fermentation and stored at −20 °C until SDS-PAGE analysis (Coomassie blue staining).

As shown in FIG. 8, higher antibody band intensity was observed in fermentations with BASI addition compared to fermentations that lacked BASI addition.

### Stabilization of Antibody a During Recovery and Storage

No BASI-containing broth was prepared using 250 g Antibody A-containing fermentation broth (produced as shown above) and 125 g sodium citrate buffer at pH 5.5. BASI containing broth was prepared using 250 g Antibody A-containing fermentation broth (produced as described above), 2.5 g BASI UF concentrate (produced as described above), and 122.5 bis-tris buffer at pH 6.5. Each broth was centrifuged at 30,000 rpm for 30 min followed by sterile filtering using a 0.2 μm Nalgene filter. Each broth was concentrated using Ultrafilter 10K MWCO. Each broth concentrate was then split into two portions with one being stored at 20 °C and one at 10 °C. SDS-PAGE analysis (Coomassie blue staining) was performed on day 1 and day 15.

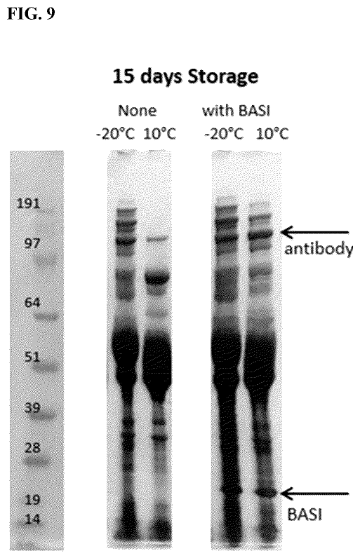

No difference in antibody band intensity was observed on day 1 (data not shown). However, on day 15, UF concentrate from broth lacking BASI addition exhibited significant loss in antibody band. With BASI addition, the antibody band remained comparable to the −20 °C reference sample (see FIG. 9).

### Recovery and Stability Profile of Antibody B Expressed in BASI Strain

The stability of UF concentrate was monitored by SDS-PAGE at 1 month by comparing −20 °C and 10 °C storage samples. At 120 h, fermentation expressing Antibody B in BASI strain was stopped, cooled to 15 °C and transferred to harvest tank. Broth was diluted with 1.5 parts of pH 5.5 50 mM sodium citrate. Diluted broth was treated with 0.033% C581 flocculant, 6% diatomaceous earth FW12, and clarified using Rotary Vacuum Drum Filter precoated with FW12 diatomaceous. The collected filtrate was concentrated using 10K MWCO UF membrane. Samples collected during each step was analyzed using SDS-PAGE for monitoring antibody stability. The obtained UF concentrate was divided into two fractions for monitoring antibody stability during storage at 10 °C and −20 °C.

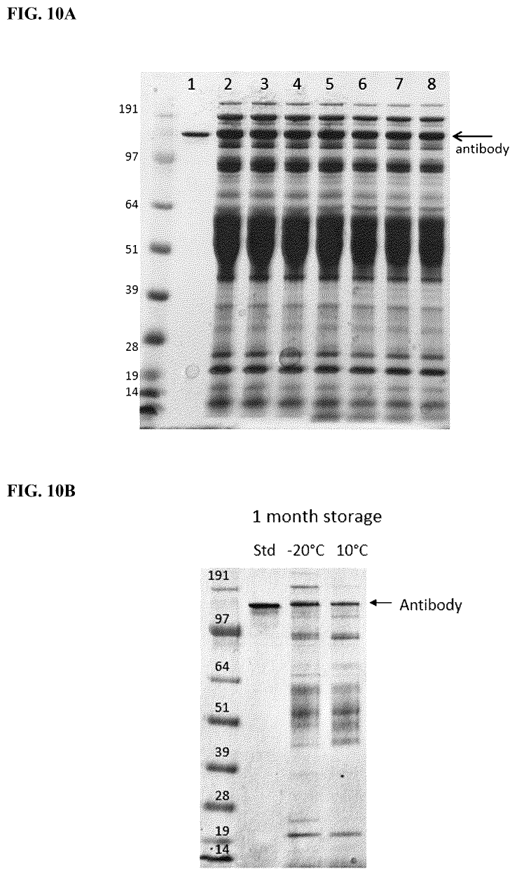

As shown in FIG. 10A, antibody bands throughout the recovery process are very similar, with no significant loss or degradation. As shown in FIG. 10B, UF concentrate derived from the BASI-expressing strain has good stability. Antibody band of sample after storage at 10 °C for 1 month is similar to the reference sample stored at −20 °C.

### Example 6: Expression of BASI in Bacillus subtilis

The yhfN region and rrnl (mutated) promoter (SEQ ID NO:3) from B. subtilis and the aprE signal sequence from B. subtilis (SEQ ID NO:4) was amplified from a B. subtilis expression strain with primers CF 17-79 and CF 19-20 (Table 7). The primer CF 17-79 also contained an overhang from the end of the alrA cassette which allowed for assembly. A 20 bp overhang from the end of the aprE signal sequence, the barley amylase subtilisin inhibitor (BASI) gene (codon optimized by the software Geneious; SEQ ID NO:5) plus 20 base pairs of the start of the BPN′ terminator from B. amyliquefaciens (SEQ ID NO:6) were synthesized by an outside vendor (Eurofins Genomics). This synthetic DNA fragment was amplified with primers CF 19-19 and CF 19-22 (Table 7). Using techniques known in the art, the two fragments were fused together using PCR with primers CF 17-79 and CF 19-22 (Table 7) to form PCR fusion 1.

TABLE 7
PCR Primers

| Primer | | |
|---|---|---|
| Name | Orientation | Sequence |
| CF 17-79 | Forward | CCTGAGATCCCTCAGGCTTTTTTTCCTCGAAATGGATCGAAGC |
| | | (SEQ ID NO: 8) |
| CF 19-20 | Reverse | CATGAACCGGAGGCGGATCTGCAGCCTGCGCAGACATGTTGC |
| | | (SEQ ID NO: 9) |
| CF 19-19 | Forward | GCAACATGTCTGCGCAGGCTGCAGATCCGCCTCCGGTTCATG |
| | | (SEQ ID NO: 10) |
| CF 19-22 | Reverse | CGGTTTTTTATGTAAGCTTATGCAGGCGGTGC (SEQ ID NO: 11) |
| CF 19-21 | Forward | GCACCGCCTGCATAAGCTTACATAAAAAACCG (SEQ ID |
| | | NO: 12) |
| CF 17-80 | Reverse | GCTTCGATCCATTTCGAGGAAAAAAAGCCTGAGGGATCTCAG |
| | | G (SEQ ID NO: 13) |

A DNA fragment containing the BPN′ terminator and the alrA expression cassette was amplified with primers CF 19-21 and CF 17-80 (Table 7; CF 17-80 has an overhang into the yhfN upstream region for future Gibson assembly) from a B. subtilis expression strain. Using techniques known in the art, this PCR fragment and PCR fusion 1 were assembled using Gibson Assembly (New England Biolabs) to create a circular DNA cassette (SEQ ID NO:7). The assembly underwent a rolling circle amplification (Evomics), which was used to transform 200 μl of competent cells of a suitable B. subtilis strain. The transformed cells were incubated at 37 °C for 1 hour while shaking at 250 rpm. This method is based on the observation that the alrA gene, which codes for alanine racemase, is essential in B. subtilis (Ferrari et al., Bio/Technol., 3:1003-1007, 1987), and thus can be used as a selectable marker. The alanine racemase converts the natural L-alanine into D-alanine that is needed for cell wall synthesis. An alanine racemase inhibitor, β-chloro-D-alanine (CDA), was used for selection/amplification of the alrA gene (Heaton et al., Biochem. Biophys. Res. Comm., 149:576-579, 1987). The alrA gene cassette was integrated with the BASI cassette into a host that was deleted for the native alrA gene and selection was performed on plates not supplemented with D-alanine. Cells from the transformation mixture were plated onto agar plates. Single colonies were selected to be grown in Luria broth with β-chloro-D-alanine (CDA) to optical density of 1.0 at 600 nm. The strain sample was then frozen at −80 °C with 20% glycerol.

A colony from this strain grown on a Luria's agar plate with the chromosomally integrated expression cassette encoding the Barley Amylase Subtilisin Inhibitor was used to inoculate 50 mL of Luria Broth in a 250 mL ultra-yield shake flask (Thomson Instrument Company). The shake flask was incubated overnight at 37 °C while rotating at 250 rpm. The entire volume of the flask was transferred to 500 mL of Bacillus culture media described below, at pH 7.3, in a 2.5 L ultra-yield shake flask (Thomson Instrument Company). The cultures were grown in a shaking incubator at 37 °C, at 150 rpm for 68 hours.

The Bacillus culture media was an enriched semi-defined media based on MOPs buffer, with urea as major nitrogen source, glucose as the main carbon source, and supplemented with 1% soytone for robust cell growth. Following growth, cells were removed by centrifugation (10,000 rpm, 45 min) and supernatant was sterilized using a Nalgene 0.2 μM filter unit.

### Example 7: Use of BASI to Stabilize Antibodies in Concentrated, Formulated Cell-Free Broth

BASI produced in Bacillus subtilis was used as a formulation ingredient to stabilize antibodies produced by Trichoderma reesei and then recovered by cell separation and concentrated by ultra-filtration. This Ultra-Filtered Concentrate (UFC) was then formulated with or without addition of BASI.

Antibody stability was assessed by SDS-PAGE followed by densitometry of the 150 kDa mature antibody band. Formulated samples were diluted to target 0.025 μg/μL initial concentration antibody. HCl was added 2:1 to denature the protein and deactivate background protease. 20 μL of 4×LDS sample buffer was added to 60 μL of the HCl-treated samples. 10 μL of each sample was loaded into the wells of a 10-well 4-12% Bis-Tris gel and then gels were run in MOPS buffer for 1 hour at 175 Volts. Gels were stained via the eStain L1 Protein Staining System and imaged using the Bio-Rad ChemiDoc MP Imaging System. Densitometry was performed using Image Lab 5.0 software.

Residual stability of the antibody was calculated by dividing the intensity of the samples stored at 4 °C or 22 °C by the intensity of samples frozen immediately after formulation (0 Days Storage).

Samples were formulated either just with buffer (A and B) or buffer and Arginine-HCl (C and D) as shown in Table 8.

TABLE 8
Sample composition

| Formulation | Composition |
|---|---|
| A | 100 mM Bis-Tris, pH 5.8 |
| B | 100 mM Bis-Tris, pH 5.8 + 3 g/kg BASI |
| C | 100 mM Bis-Tris, pH 5.8 + 2.3% Arginine-HCl |
| D | 100 mM Bis-Tris, pH 5.8 + 2.3% Arginine-HCl + 3 g/kg |
| | BASI |

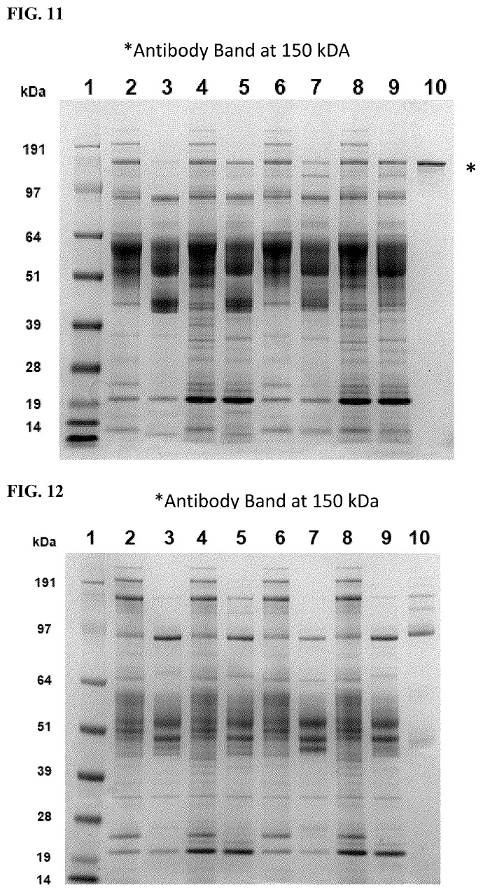

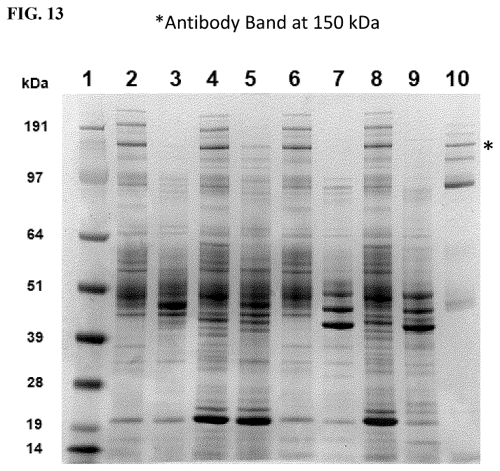

Stability analysis for antibodies produced in three separate fermentations are shown in FIG. 11. FIG. 12, and FIG. 13.

As shown in FIG. 11, Antibody B fermented at 14L scale at pH5.5 (UFC 20200577) and stored at 4 °C for 56 days showed that addition of BASI increased the Residual Band Density from 7.1% to 45.2% when using just Bis-Tris buffer. When stored buffered in the presence of 2.3% Arginine-HCl, addition of BASI increased Residual Band Density from 27.9% to 79.8%. As shown in FIG. 12, Antibody B fermented at 500L scale at pH7 (UFC 20208053) and stored at 22 °C for 25 days showed that addition of BASI increased the Residual Band Density from 0.7% to 7.6% when using just buffer Bis-Tris. When buffered in the presence of 2.3% Arginine-HCl, addition of BASI increased Residual Band Density from 4.6% to 22.6%. As shown in FIG. 13, Antibody A fermented at 14L scale at pH6.5 (UFC 20200874) and stored at 22 °C for 25 days showed that addition of BASI increased the Residual Band Density from 0% to 1.6% when using just buffer Bis-Tris. When buffered in the presence of 2.3% Arginine-HCl, addition of BASI increased Residual Band Density from 0% to 14.6%. Table 9 summarizes the change in stability observed with the addition of BASI to antibody UFCs under different conditions.

TABLE 9
Summary of changes in stability with BASI addition

| UFC | UFC 20200577 | UFC 20208053 | UFC 20200874 |
|---|---|---|---|
| Antibody | B | B | A |
| Fermentation | 14 L, pH 5.5 | 500 L, pH 7.0 | 14 L, pH 6.5 |
| Storage | 4 °C, 56 days | 22 °C, 25 days | 22 °C, 25 days |
| | % Residual Band Density | | |
| Formulation A | 7.1 | 0.7 | 0 |
| Formulation B | 45.2 | 7.6 | 1.6 |
| Formulation C | 27.9 | 4.6 | 0 |
| Formulation D | 79.8 | 22.6 | 14.6 |

### REFERENCES

Buckland, B., Brix, T., Fastert, H., Gbewonyo, K., Hunt, G., & Jain, D. (1985). Fermentation Exhaust Gas Analysis Using Mass Spectrometry. Bio/Technology (3), 982-988.
Jørgensen, M. S., Skovlund, D. A., Johannesen, P. F. et al. A novel platform for heterologous gene expression in Trichoderma reesei (Teleomorph Hypocrea jecorina). Microb Cell Fact 13, 33 (2014).
Sheir-Neiss, G., Montenecourt, B. S. Characterization of the secreted cellulases of Trichoderma reesei wild type and mutants during controlled fermentations. Appl Microbiol Biotechnol 20, 46-53 (1984).

SEQUENCES

| SEQ ID | | |
|---|---|---|
| NO | SEQUENCE | DESCRIPTION |
| 1 | GCGGACCCTCCGCCTGTCCATGATACTGATGGCCACGAGCTCC | BASI |
| | GAGCGGACGCCAACTACTATGTGCTGAGCGCCAACCGAGCCCA | (predicted |
| | CGGCGGAGGCCTCACCATGGCCCCCGGCCATGGCCGCCACTGC | mature) |
| | CCCCTGTTCGTGTCCCAAGACCCTAACGGCCAGCACGACGGCT | |
| | TTCCGGTTCGCATCACCCCCTACGGTGTCGCCCCTTCCGACAAG | |
| | ATTATCCGCCTCAGCACGGACGTTCGCATTAGCTTCCGAGCGT | |
| | ACACTACGTGCCTCCAGAGCACGGAGTGGCACATCGACAGCG | |
| | AGCTCGCCGCGGGACGCCGACACGTGATTACCGGCCCTGTTAA | |
| | GGATCCCAGCCCCTCGGGTCGAGAGAATGCTTTCCGCATCGAG | |
| | AAGTACTCCGGCGCGGAGGTCCACGAGTACAAGCTGATGAGCT | |
| | GCGGCGACTGGTGCCAAGACCTGGGCGTTTTCCGCGACCTGAA | |
| | GGGCGGTGCGTGGTTTCTCGGCGCCACCGAGCCTTACCACGTG | |
| | GTCGTGTTTAAGAAGGCCCCTCCTGCCTAA | |
| 2 | CTACAACATCGTCCTGCAGTTCCTATATGATACGTATAACCCAT | cbh2 |
| | TGATACATCCAAGTATCCAGAGGTGTATGGAAATATCAGATCA | promoter- |
| | AGACCTCTCTCTTCTAAGAAACCTAGAACCAGACGCTGGTAGT | CBH1 |
| | ATAATAAGCACACTGTGACTCGCTTAGGCCCTTAAGCTTAGGC | core/linker- |
| | CGGCTTGCTTACTATTAACCTCTCATAAACGCTACTGCAATGAT | BASI-trpC |
| | TGGAAACTTCTTATAGTAGAATGAGGCAATAAGACGCATCTCA | terminator |
| | GGTCACATATAGTCTTATGTTTGAAACCCCTCACTACTGCCATT | expression |
| | TATCTTGTGGAAATATCTATTATTTCAGTCTATACGTAATGAAG | cassette |
| | GCACTTTTCAGGATCTCTTCCCTAAGCTTGTATAAGCAGGTTTG | |
| | TTGCCGTAACCATTCTGTCTCCTCGCCTAATACCTGTGAAGCAC | |
| | AGAATACGTTTATTCTATAAGAGACGTCTTACCTTCCATCGAG | |
| | ATTGAAAGCTTAAACCGTCTACAACGGATGCCCTCATCATGAC | |
| | CCGTCTAACTCGAACATCTGCCACATTAGTCTCGGGTAACAGG | |
| | AGGAGTAACACGACCAGTGTAACACGTTAAGCATACAATTGA | |
| | ACGAGAATGGTGAGGACTGAGATAAAAGAATTCTGTTAAGGA | |
| | TCTAAAATTATAGTGCATACAAGGTAGATGTTAGTAGGTGGTT | |
| | TCAGTTTTCCTTTCCTTTACGTTGGTATAGAGCAGCGTTCACCA | |
| | AATGTTAGCAGAGTTCTATCTATGTCGTATCCATTCTGCCTTAT | |
| | ATCTCTCAAGGGCGCCGAGCTCATCCTACGAAGCTCTCAGGCC | |
| | ATCGTAGGAAATACAGGATAGACACTGAATTCTAGGCTAGGTA | |
| | TGCGAGGCACGCGGATCTAGGGCAGACTGGGCATTGCATAGCT | |
| | ATGGTGTAGTAGAACTCCCGTCAACGGCTATTCTCACCTAGAC | |
| | TTTCCCCTTCGAACTGACAAGTTGTTATATTGCCTGTGTACCAA | |
| | GCGCTAATGTGGACAGGATTAATGCCAGAGTTCATTAGCCTCA | |
| | AGTAGAGCCTATTTCCTCGCCGGAAAGTCATCTCTCTTATTGCA | |
| | TTTCTGCCCTTCCCACTAACTCAGGGTGCAGCGCAACACTACA | |
| | CGCAACATATACACTTTATTAGCCGTGCAACAAGGCTATTCTA | |
| | CGAAAAATGCTACACTCCACATGTTAAAGGCGCATTCAACCAG | |
| | CTTCTTTATTGGGTAATATACAGCCAGGCGGGGATGAAGCTCA | |
| | TTAGCCGCCACTCAAGGCTATACAATGTTGCCAACTCTCCGGG | |
| | CTTTATCCTGTGCTCCCGAATACCACATCGTGATGATGCTTCAG | |
| | CGCACGGAAGTCACAGACACCGCCTGTATAAAAGGGGGACTG | |
| | TGACCCTGTATGAGGCGCAACATGGTCTCACAGCAGCTCACCT | |
| | GAAGAGGCTTGTAAGATCACCCTCTGTGTATTGCACCATGTAT | |
| | CGGAAGTTGGCCGTCATCTCGGCCTTCTTGGCCACAGCTCGTG | |
| | CTCAGTCGGCCTGCACTCTCCAATCGGAGACTCACCCGCCTCT | |
| | GACATGGCAGAAATGCTCGTCTGGTGGCACGTGCACTCAACAG | |
| | ACAGGCTCCGTGGTCATCGACGCCAACTGGCGCTGGACTCACG | |
| | CTACGAACAGCAGCACGAACTGCTACGATGGCAACACTTGGA | |
| | GCTCGACCCTATGTCCTGACAACGAGACCTGCGCGAAGAACTG | |
| | CTGTCTGGACGGTGCCGCCTACGCGTCCACGTACGGAGTTACC | |
| | ACGAGCGGTAACAGCCTCTCCATTGGCTTTGTCACCCAGTCTG | |
| | CGCAGAAGAACGTTGGCGCTCGCCTTTACCTTATGGCGAGCGA | |
| | CACGACCTACCAGGAATTCACCCTGCTTGGCAACGAGTTCTCT | |
| | TTCGATGTTGATGTTTCGCAGCTGCCGTAAGTGACTTACCATGA | |
| | ACCCCTGACGCTATCTTCTTGTTGGCTCCCAGCTGACTGGCCAA | |
| | TTCAAGGTGCGGCTTGAACGGAGCTCTCTACTTCGTGTCCATG | |
| | GACGCGGATGGTGGCGTGAGCAAGTATCCCACCAACACCGCTG | |
| | GCGCCAAGTACGGCACGGGGTACTGTGACAGCCAGTGTCCCCG | |
| | CGATCTGAAGTTCATCAATGGCCAGGCCAACGTTGAGGGCTGG | |
| | GAGCCGTCATCCAACAACGCGAACACGGGCATTGGAGGACAC | |
| | GGAAGCTGCTGCTCTGAGATGGATATCTGGGAGGCCAACTCCA | |
| | TCTCCGAGGCTCTTACCCCCCACCCTTGCACGACTGTCGGCCAG | |
| | GAGATCTGCGAGGGTGATGGGTGCGGCGGAACTTACTCCGATA | |
| | ACAGATATGGCGGCACTTGCGATCCCGATGGCTGCGACTGGAA | |
| | CCCATACCGCCTGGGCAACACCAGCTTCTACGGCCCTGGCTCA | |
| | AGCTTTACCCTCGATACCACCAAGAAATTGACCGTTGTCACCC | |
| | AGTTCGAGACGTCGGGTGCCATCAACCGATACTATGTCCAGAA | |
| | TGGCGTCACTTTCCAGCAGCCCAACGCCGAGCTTGGTAGTTAC | |
| | TCTGGCAACGAGCTCAACGATGATTACTGCACAGCTGAGGAGG | |
| | CAGAATTCGGCGGATCCTCTTTCTCAGACAAGGGCGGCCTGAC | |
| | TCAGTTCAAGAAGGCTACCTCTGGCGGCATGGTTCTGGTCATG | |
| | AGTCTGTGGGATGATGTGAGTTTGATGGACAAACATGCGCGTT | |
| | GACAAAGAGTCAAGCAGCTGACTGAGATGTTACAGTACTACGC | |
| | CAACATGCTGTGGCTGGACTCCACCTACCCGACAAACGAGACC | |
| | TCCTCCACACCCGGTGCCGTGCGCGGAAGCTGCTCCACCAGCT | |
| | CCGGTGTCCCTGCTCAGGTCGAATCTCAGTCTCCCAACGCCAA | |
| | GGTCACCTTCTCCAACATCAAGTTCGGACCCATTGGCAGCACC | |
| | GGCAACCCTAGCGGCGGCAACCCTCCCGGCGGAAACCCGCCTG | |
| | GCACCACCACCACCCGCCGCCCAGCCACTACCACTGGAAGCTC | |
| | TCCCGGACCTACTAAGGTCGCCGTTGAGAAACGCGCGGACCCT | |
| | CCGCCTGTCCATGATACTGATGGCCACGAGCTCCGAGCGGACG | |
| | CCAACTACTATGTGCTGAGCGCCAACCGAGCCCACGGCGGAGG | |
| | CCTCACCATGGCCCCCGGCCATGGCCGCCACTGCCCCCTGTTC | |
| | GTGTCCCAAGACCCTAACGGCCAGCACGACGGCTTTCCGGTTC | |
| | GCATCACCCCCTACGGTGTCGCCCCTTCCGACAAGATTATCCG | |
| | CCTCAGCACGGACGTTCGCATTAGCTTCCGAGCGTACACTACG | |
| | TGCCTCCAGAGCACGGAGTGGCACATCGACAGCGAGCTCGCCG | |
| | CGGGACGCCGACACGTGATTACCGGCCCTGTTAAGGATCCCAG | |
| | CCCCTCGGGTCGAGAGAATGCTTTCCGCATCGAGAAGTACTCC | |
| | GGCGCGGAGGTCCACGAGTACAAGCTGATGAGCTGCGGCGAC | |
| | TGGTGCCAAGACCTGGGCGTTTTCCGCGACCTGAAGGGCGGTG | |
| | CGTGGTTTCTCGGCGCCACCGAGCCTTACCACGTGGTCGTGTTT | |
| | AAGAAGGCCCCTCCTGCCTAAGTGTCGATGTCAGCTCCGGAGT | |
| | TGAGACAAATGGTGTTCAGGATCTCGATAAGATACGTTCATTT | |
| | GTCCAAGCAGCAAAGAGTGCCTTCTAGTGATTTAATAGCTCCA | |
| | TGTCAACAAGAATAAAACGCGTTTCGGGTTTACCTCTTCCAGA | |
| | TACAGCTCATCTGCAATGCATTAATGCATTGGACCTCGCAACC | |
| | CTAGTACGCCCTTCAGGCTCCGGCGAAGCAGAAGAATAGCTTA | |
| | GCAGAGTCTATTTTCATTTTCGGGAGACGAGATCAAGCAGATC | |
| | AACGGTCGTCAAGAGACCTACGAGACTGAGGAATCCGCTCTTG | |
| | GCTCCACGCGACTATATATTTGTCTCTAATTGTACTTTGACATG | |
| | CTCCTCTTCTTAACTCTGATAGCTTGACTATGAAAATTCCGTCA | |
| | CCAGCCCCTGGGTTCGCAAAGATAATTGCACTGTTTCTTCCTTG | |
| | AACTCTCAAGCCTACAGGACACACATTCATCGTAGGTATAAAC | |
| | CTCGAAAATCATTCCTACTAAGATGGGTATACAATAGTAACCA | |
| | TGGTTGCCTAGTGAATGCTCCGTAACACCCAATACGCCGGCCG | |
| | AAACTTTTTTACAACTCTCCTATGAGTCGTTTACCCAGAATGCA | |
| | CAGGTACACTTGTTTAGAGG | |
| 3 | GCTGATAAACAGCTGACATCAACTAAAAGTTTCATTAAA | rrnI |
| | TACTTTGAAAAAAGTTGTTGACTTAAAAGAAGCTAAATG | promoter |
| | TTATAGTAATTGTACAGAATAGTCTTTTAAGTAAGTCTAC | from B. |
| | TCTGAATTTTTTTAAAAGGAGAGGGTAAAGA | subtilis |
| 4 | GTGAGAAGCAAAAAATTGTGGATCAGCTTGTTGTTTGCGTTAA | aprE signal |
| | CGTTAATCTTTACGATGGGTTCAGCAACATGTCTGCTAGCGCA | peptide |
| | | from B. |
| | | subtilis |
| 5 | GCAGATCCGCCTCCGGTTCATGATACAGATGGCCATGAA | Codon |
| | CTGAGAGCAGATGCAAACTATTATGTTCTGTCAGCAAAT | optimized |
| | AGAGCACATGGCGGAGGCCTGACAATGGCACCTGGCCAT | BASI gene |
| | GGCAGACATTGTCCGCTGTTTGTTTCACAAGATCCGAATG | |
| | GCCAACATGATGGCTTTCCGGTTAGAATTACACCGTATGG | |
| | CGTTGCACCGTCAGATAAAATCATTAGACTGAGCACAGA | |
| | TGTCCGCATTAGCTTTAGAGCATATACAACATGCCTGCAA | |
| | TCAACGGAATGGCATATTGATTCAGAACTGGCAGCAGGC | |
| | AGACGCCATGTTATTACAGGACCGGTTAAAGATCCGTCA | |
| | CCGTCAGGCAGAGAAAATGCGTTTAGAATCGAAAAATAC | |
| | AGCGGAGCGGAAGTCCATGAATATAAACTGATGTCATGC | |
| | GGAGATTGGTGCCAAGATCTGGGCGTTTTTAGAGATCTG | |
| | AAAGGCGGAGCATGGTTTCTTGGCGCAACAGAACCGTAT | |
| | CATGTTGTCGTTTTCAAAAAAGCACCGCCTGCA(TAA) | |
| 6 | TACATAAAAAACCGGCCTTGGCCCCGCCGGTTTTTTATTA | B. |
| | TTTTTCTTCCTCCGCATGTTCAATCCGCTCCATAATCGACG | amyliquefaciens |
| | GATGGCTCCCTCTGAAAATTTTAACGAGAAACGGCGGGT | terminator |
| | TGACCCGGCTCAGTCCCGTAACGGCCAAGTCCTGAAACG | |
| | TCTCAATCGCCGCTTCCCGGTTTCCGGTCAGCTCAATGCC | |
| | GTAACGGTCGGCGGCGTTTTCCTGATACCGGGAGACGGC | |
| | ATTCGTAATC | |
| 7 | ATGAGCACAAAACCTTTTTACAGAGATACGTGGGCGGAA | B. subtilis |
| | ATTGACTTGTCCGCGATAAAGGAAAATGTCAGCAATATG | alrA gene |
| | AAAAAACATATCGGTGAACATGTCCACTTGATGGCAGTT | expression |
| | GTGAAAGCAAACGCCTACGGGCATGGTGATGCAGAAACA | cassette |
| | GCAAAGGCTGCTCTTGACGCAGGTGCTTCATGCTTGGCCG | |
| | TGGCCATTTTGGATGAAGCGATTTCACTGCGCAAAAAGG | |
| | GATTGAAGGCGCCTATATTGGTGCTTGGCGCGGTTCCCCC | |
| | GGAGTATGTGGCAATCGCTGCTGAGTATGACGTGACCTT | |
| | AACAGGTTATTCTGTTGAATGGCTTCAGGAGGCAGCCCG | |
| | CCACACGAAAAAAGGTTCTCTTCATTTTCATCTGAAGGTC | |
| | GATACGGGGATGAACAGACTTGGTGTAAAAACAGAGGA | |
| | AGAAGTTCAGAACGTGATGGCAATTCTTGACCGCAACCC | |
| | TCGTTTAAAGTGCAAAGGGGTATTTACCCATTTTGCGACA | |
| | GCGGATGAAAAAGAAAGAGGCTATTTCTTAATGCAGTTT | |
| | GAGCGCTTTAAAGAGCTGATTGCTCCGCTGCCGTTAAAG | |
| | AATCTAATGGTCCACTGCGCGAACAGCGCCGCTGGACTC | |
| | CGGCTGAAAAAAGGCTTTTTTAATGCAGTCAGATTCGGC | |
| | ATCGGCATGTATGGCCTTCGCCCGTCTGCTGACATGTCGG | |
| | ACGAGATACCGTTTCAGCTGCGTCCGGCATTTACCCTGCA | |
| | TTCGACACTGTCACATGTCAAACTGATCAGAAAAGGCGA | |
| | GAGCGTCAGCTACGGAGCCGAGTACACAGCGGAAAAAG | |
| | ACACATGGATCGGGACGGTGCCTGTAGGCTATGCGGACG | |
| | GCTGGCTCCGAAAATTGAAAGGGACCGACATCCTTGTGA | |
| | AGGGAAAACGCCTGAAAATTGCCGGCCGAATTTGCATGG | |
| | ACCAATTTATGGTGGAGCTGGATCAGGAATATCCGCCGG | |
| | GCACAAAAGTCACATTAATAGGCCGGCAGGGGGATGAAT | |
| | ATATTTCCATGGATGAGATTGCAGGAAGGCTCGAAACCA | |
| | TTAACTATGAGGTGGCCTGTACAATAAGTTCCCGTGTTCC | |
| | CCGTATGTTTTTGGAAAATGGGAGTATAATGGAAGTAAG | |
| | AAATCCTTTATTGCAGGTAAATATAAGCAATTAACTTACC | |
| | TAAAAAGCCTGAGATCCCTCAGGCTTTTTTTCCTC | |
| 8 | CCTGAGATCCCTCAGGCTTTTTTTCCTCGAAATGGATCGA | CF 17-79 |
| | AGC | forward |
| | | primer |
| 9 | CATGAACCGGAGGCGGATCTGCAGCCTGCGCAGACATGT | CF 19-20 |
| | TGC | Reverse |
| | | primer |
| 10 | GCAACATGTCTGCGCAGGCTGCAGATCCGCCTCCGGTTCA | CF 19-19 |
| | TG | Forward |
| | | primer |
| 11 | CGGTTTTTTATGTAAGCTTATGCAGGCGGTGC | CF 19-22 |
| | | Reverse |
| | | primer |
| 12 | GCACCGCCTGCATAAGCTTACATAAAAAACCG | CF 19-21 |
| | | Forward |
| | | primer |
| 13 | GCTTCGATCCATTTCGAGGAAAAAAAGCCTGAGGGATCT | CF 17-80 |
| | CAGG | Reverse |
| | | primer |

We claim:

1. A recombinant cell comprising a) a heterologously expressed barley alpha-amylase subtilisin inhibitor (BASI) polypeptide; and b) a heterologously expressed monoclonal antibody or functional fragment thereof.

2. The cell of claim 1, wherein the antibody or functional fragment thereof is a therapeutic antibody or functional fragment thereof.

3. The cell of claim 1 or claim 2, wherein the cell is a bacterial, fungal, yeast, plant, or mammalian cell.

4. The cell of claim 3, wherein the cell is a Trichoderma reesei cell or an Aspergillus niger cell.

5. The cell of claim 3, wherein the cell is a Bacillus subtilis cell.

6. The cell of claim 3, wherein the cell is a Chinese Hamster Ovary (CHO) or human embryonic kidney (HEK) cell.

7. The cell of any one of claims 1-6, wherein the BASI polypeptide comprises the amino acid sequence of SEQ ID NO:1.

8. The cell of any one of claims 1-7, wherein the antibody or functional fragment thereof exhibits less proteolytic degradation compared to an antibody or functional fragment thereof that is not heterologously co-expressed with a BASI polypeptide.

9. The cell of any one of claims 1-8, wherein the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.

10. A fermentation broth comprising the cell of any one of claims 1-9.

11. A method for decreasing proteolysis of a heterologously expressed monoclonal antibody or functional fragment thereof comprising culturing a recombinant cell comprising a) a heterologously expressed barley alpha-amylase subtilisin inhibitor (BASI) polypeptide; and b) the heterologously expressed monoclonal antibody or functional fragment thereof under suitable conditions for production of the heterologously expressed antibody or functional fragment thereof and the BASI polypeptide.

12. The method of claim 11, further comprising isolating the antibody or functional fragment thereof.

13. The method of claim 11 or claim 12, wherein the antibody or functional fragment thereof is a therapeutic antibody or functional fragment thereof.

14. The method of any one of claims 11-13, wherein the cell is a bacterial, fungal, yeast, mammalian, or plant cell.

15. The method of claim 14, wherein the cell is a Trichoderma reesei cell or an Aspergillus niger cell.

16. The method of claim 14, wherein the cell is a Bacillus subtilis cell.

17. The method of claim 14, wherein the cell is a Chinese Hamster Ovary (CHO) or human embryonic kidney (HEK) cell.

18. The method of any one of claims 11-17, wherein the BASI polypeptide comprises the amino acid sequence of SEQ ID NO:1.

19. The method of any one of claims 11-18, wherein the antibody or functional fragment thereof exhibits less proteolytic degradation compared to an antibody or functional fragment thereof that is not heterologously co-expressed with a BASI polypeptide.

20. The method of any one of claims 11-19, wherein the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.

21. A method for decreasing proteolysis of a recombinantly expressed monoclonal antibody or functional fragment thereof comprising isolating the recombinantly expressed antibody or functional fragment thereof in the presence of an exogenously added barley alpha-amylase subtilisin inhibitor (BASI) polypeptide.

22. The method of claim 21, wherein the antibody or functional fragment thereof is a therapeutic antibody or functional fragment thereof.

23. The method of claim 21 or claim 22, wherein the BASI polypeptide is recombinantly expressed in a bacterial, fungal, yeast, mammalian, or plant cell.

24. The method of any one of claims 21-23, wherein the BASI polypeptide is recombinantly expressed in a Bacillus subtilis cell or an Aspergillus niger cell.

25. The method of any one of claims 21-24, wherein the BASI polypeptide comprises the amino acid sequence of SEQ ID NO:1.

26. The method of any one of claims 21-25, wherein the monoclonal antibody or functional fragment thereof is recombinantly expressed in a bacterial, fungal, yeast, mammalian, or plant cell.

27. The method of claim 26, wherein the monoclonal antibody or functional fragment thereof is recombinantly expressed in a Trichoderma reesei cell or an Aspergillus niger cell.

28. The method of claim 26, wherein the monoclonal antibody or functional fragment thereof is recombinantly expressed in a Bacillus subtilis cell.

29. The method of claim 26, wherein the monoclonal antibody or functional fragment thereof is recombinantly expressed in a Chinese Hamster Ovary (CHO) or human embryonic kidney (HEK) cell.

30. The method of any one of claims 21-29, wherein the antibody or functional fragment thereof exhibits less proteolytic degradation and/or improved yields of intact protein compared to an antibody or functional fragment thereof that is not isolated in the presence of an exogenously added BASI polypeptide.

31. The method of any one of claims 21-30, wherein the functional fragment is selected from the group consisting of Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the same.
